SEQUENCE LISTING (194 sequences)

SEQ ID NO: 1 - protein, 11 aa, Artificial Sequence
  Synthetic
QASQNIYTSLA

SEQ ID NO: 2 - protein, 7 aa, Artificial Sequence
  Synthetic
GASNLAS

SEQ ID NO: 3 - protein, 12 aa, Artificial Sequence
  Synthetic
  misc_feature (11)..(12): Xaa can be any naturally occurring amino acid
LGSYTQIDTTXX

SEQ ID NO: 4 - protein, 5 aa, Artificial Sequence
  Synthetic
TYGMT

SEQ ID NO: 5 - protein, 16 aa, Artificial Sequence
  Synthetic
IIVSSGNTYYANWAQG

SEQ ID NO: 6 - protein, 17 aa, Artificial Sequence
  Synthetic
  misc_feature (15)..(17): Xaa can be any naturally occurring amino acid
GLGRAYSSGYGMDGXXX

SEQ ID NO: 7 - protein, 11 aa, Artificial Sequence
  Synthetic
QASQSIRSWLS

SEQ ID NO: 8 - protein, 7 aa, Artificial Sequence
  Synthetic
EASKLAS

SEQ ID NO: 9 - protein, 12 aa, Artificial Sequence
  Synthetic
  misc_feature (11)..(12): Xaa can be any naturally occurring amino acid
LGSIASDDTAXX

SEQ ID NO: 10 - protein, 5 aa, Artificial Sequence
  Synthetic
RYTMA

SEQ ID NO: 11 - protein, 16 aa, Artificial Sequence
  Synthetic
WIDSSGSTYYASWAKG

SEQ ID NO: 12 - protein, 17 aa, Artificial Sequence
  Synthetic
  misc_feature (15)..(17): Xaa can be any naturally occurring amino acid
GNDGGDPLYYGMDPXXX

SEQ ID NO: 13 - protein, 11 aa, Artificial Sequence
  Synthetic
QASESIYSDLA

SEQ ID NO: 14 - protein, 7 aa, Artificial Sequence
  Synthetic
FASTLAS

SEQ ID NO: 15 - protein, 12 aa, Artificial Sequence
  Synthetic
  misc_feature (10)..(12): Xaa can be any naturally occurring amino acid
LGDYGNSWAXXX

SEQ ID NO: 16 - protein, 5 aa, Artificial Sequence
  Synthetic
SYSMG

SEQ ID NO: 17 - protein, 16 aa, Artificial Sequence
  Synthetic
IIYNTGRIDYANWAKG

SEQ ID NO: 18 - protein, 17 aa, Artificial Sequence
  Synthetic
  misc_feature (7)..(17): Xaa can be any naturally occurring amino acid
GYPGDIXXXXXXXXXXX

SEQ ID NO: 19 - protein, 11 aa, Artificial Sequence
  Synthetic
QASQSIGSYLS

SEQ ID NO: 20 - protein, 7 aa, Artificial Sequence
  Synthetic
QASKMAS

SEQ ID NO: 21 - protein, 12 aa, Artificial Sequence
  Synthetic
  misc_feature (10)..(12): Xaa can be any naturally occurring amino acid
LGHYVGDWSXXX

SEQ ID NO: 22 - protein, 5 aa, Artificial Sequence
  Synthetic
TYAMI

SEQ ID NO: 23 - protein, 16 aa, Artificial Sequence
  Synthetic
IISYGGSAYYASWAKG

SEQ ID NO: 24 - protein, 17 aa, Artificial Sequence
  Synthetic
DRGFGYGYGIGYYGLDP

SEQ ID NO: 25 - protein, 11 aa, Artificial Sequence
  Synthetic
LASEDIYNTIS

SEQ ID NO: 26 - protein, 7 aa, Artificial Sequence
  Synthetic
GASNLKS

SEQ ID NO: 27 - protein, 12 aa, Artificial Sequence
  Synthetic
LGGYSYSSTGLT

SEQ ID NO: 28 - protein, 5 aa, Artificial Sequence
  Synthetic
SYAMT

SEQ ID NO: 29 - protein, 16 aa, Artificial Sequence
  Synthetic
VIGTSGMRYYANWAKG

SEQ ID NO: 30 - protein, 17 aa, Artificial Sequence
  Synthetic
  misc_feature (17)..(17): Xaa can be any naturally occurring amino acid
ANFYVGNDESTDAFDPX

SEQ ID NO: 31 - protein, 11 aa, Artificial Sequence
  Synthetic
QASQNIGRWLA

SEQ ID NO: 32 - protein, 7 aa, Artificial Sequence
  Synthetic
RASDLAS

SEQ ID NO: 33 - protein, 12 aa, Artificial Sequence
  Synthetic
  misc_feature (11)..(12): Xaa can be any naturally occurring amino acid
LGSYSSSDSAXX

SEQ ID NO: 34 - protein, 5 aa, Artificial Sequence
  Synthetic
TYTLG

SEQ ID NO: 35 - protein, 16 aa, Artificial Sequence
  Synthetic
WIDSSGSAYYASWAKG

SEQ ID NO: 36 - protein, 17 aa, Artificial Sequence
  Synthetic
  misc_feature (15)..(17): Xaa can be any naturally occurring amino acid
GNTGGDPLYYGMDPXXX

SEQ ID NO: 37 - protein, 13 aa, Artificial Sequence
  Synthetic
  misc_feature (13)..(13): Xaa can be any naturally occurring amino acid
TLSSAHKTYSIGX

SEQ ID NO: 38 - protein, 9 aa, Artificial Sequence
  Synthetic
LRRDGSYTK

SEQ ID NO: 39 - protein, 13 aa, Artificial Sequence
  Synthetic
  misc_feature (10)..(13): Xaa can be any naturally occurring amino acid
GVDYDGGYVXXXX

SEQ ID NO: 40 - protein, 5 aa, Artificial Sequence
  Synthetic
NYIIT

SEQ ID NO: 41 - protein, 16 aa, Artificial Sequence
  Synthetic
MISTDGDTYYANWAKG

SEQ ID NO: 42 - protein, 17 aa, Artificial Sequence
  Synthetic
  misc_feature (8)..(17): Xaa can be any naturally occurring amino acid
GLTNFFIXXXXXXXXXX

SEQ ID NO: 43 - protein, 13 aa, Artificial Sequence
  Synthetic
  misc_feature (13)..(13): Xaa can be any naturally occurring amino acid
TLSSAHSTYTIAX

SEQ ID NO: 44 - protein, 9 aa, Artificial Sequence
  Synthetic
LKSDGSYTR

SEQ ID NO: 45 - protein, 13 aa, Artificial Sequence
  Synthetic
  misc_feature (10)..(13): Xaa can be any naturally occurring amino acid
GADYSGGYLXXXX

SEQ ID NO: 46 - protein, 5 aa, Artificial Sequence
  Synthetic
AYAMT

SEQ ID NO: 47 - protein, 16 aa, Artificial Sequence
  Synthetic
MISTDGATYYASWAKG

SEQ ID NO: 48 - protein, 17 aa, Artificial Sequence
  Synthetic
  misc_feature (8)..(17): Xaa can be any naturally occurring amino acid
GLTNFFIXXXXXXXXXX

SEQ ID NO: 49 - protein, 13 aa, Artificial Sequence
  Synthetic
  misc_feature (13)..(13): Xaa can be any naturally occurring amino acid
TLSSAHKTYTIAX

SEQ ID NO: 50 - protein, 13 aa, Artificial Sequence
  Synthetic
  misc_feature (10)..(13): Xaa can be any naturally occurring amino acid
GADYNGGYVXXXX

SEQ ID NO: 51 - protein, 5 aa, Artificial Sequence
  Synthetic
SSAIT

SEQ ID NO: 52 - protein, 16 aa, Artificial Sequence
  Synthetic
MISTDGATYYANWAKG

SEQ ID NO: 53 - protein, 17 aa, Artificial Sequence
  Synthetic
  misc_feature (8)..(17): Xaa can be any naturally occurring amino acid
GLTNFSIXXXXXXXXXX

SEQ ID NO: 54 - protein, 13 aa, Artificial Sequence
  Synthetic
  misc_feature (12)..(13): Xaa can be any naturally occurring amino acid
TADTLSRTYASXX

SEQ ID NO: 55 - protein, 9 aa, Artificial Sequence
  Synthetic
  misc_feature (8)..(9): Xaa can be any naturally occurring amino acid
RDTSRPSXX

SEQ ID NO: 56 - protein, 13 aa, Artificial Sequence
  Synthetic
ATSDGSGSSYQYV

SEQ ID NO: 57 - protein, 5 aa, Artificial Sequence
  Synthetic
SYHMS

SEQ ID NO: 58 - protein, 16 aa, Artificial Sequence
  Synthetic
IIYFTGDASYASWAKG

SEQ ID NO: 59 - protein, 17 aa, Artificial Sequence
  Synthetic
  misc_feature (15)..(17): Xaa can be any naturally occurring amino acid
DTDWNSSRTIPFDPXXX

SEQ ID NO: 60 - protein, 13 aa, Artificial Sequence
  Synthetic
KCDSGNIDNYYVH

SEQ ID NO: 61 - protein, 9 aa, Artificial Sequence
  Synthetic
  misc_feature (8)..(9): Xaa can be any naturally occurring amino acid
NDDQRPSXX

SEQ ID NO: 62 - protein, 13 aa, Artificial Sequence
  Synthetic
  misc_feature (11)..(13): Xaa can be any naturally occurring amino acid
LSSVSSDNYVXXX

SEQ ID NO: 63 - protein, 5 aa, Artificial Sequence
  Synthetic
SYAMS

SEQ ID NO: 64 - protein, 16 aa, Artificial Sequence
  Synthetic
IIYPNDITYYASWAKG

SEQ ID NO: 65 - protein, 17 aa, Artificial Sequence
  Synthetic
  misc_feature (16)..(17): Xaa can be any naturally occurring amino acid
VVSWPGYTTEYYFNIXX

SEQ ID NO: 66 - protein, 110 aa, Artificial Sequence
  Synthetic
  misc_feature (99)..(100): Xaa can be any naturally occurring amino acid
DPVLTQTPSSTSAAVGGTVTIKCQASQNIYTSLAWYQQKPGQPPKLLIYGASNLASGVPSRFKGSRSGTEYTLTISGVEREDAATYYCLGSYTQIDTTXXFGGGTKLEIK

SEQ ID NO: 67 - protein, 123 aa, Artificial Sequence
  Synthetic
  misc_feature (110)..(112): Xaa can be any naturally occurring amino acid
QKQLVESGGRLVTPGGSLTLTCTISGIDLNTYGMTWVRQAPGKGLEYIGIIVSSGNTYYANWAQGRFTISKTSTTVDLKMTSLTASDTATYFCARGLGRAYSSGYGMDGXXXWGPGTLVTVSS

SEQ ID NO: 68 - protein, 110 aa, Artificial Sequence
  Synthetic
  misc_feature (99)..(100): Xaa can be any naturally occurring amino acid
DGVMTQTPSSTSAAVGGTVTIKCQASQSIRSWLSWYQQKPGQPPKLLIYEASKLASGVPSRFKGSRSGTEYTLAISGVQREDAATYYCLGSIASDDTAXXFGAGTNVEIK

SEQ ID NO: 69 - protein, 124 aa, Artificial Sequence
  Synthetic
  misc_feature (3)..(3): Xaa can be any naturally occurring amino acid
  misc_feature (111)..(113): Xaa can be any naturally occurring amino acid
QSXLEESGGRLVTPGTPLTLTCTVSGFSLSRYTMAWVRQAPGKGLEYIGWIDSSGSTYYASWAKGRFTISKTSSTTVDLKMTSLTTEDTATYFCASGNDGGDPLYYGMDPXXXWGPGTLVTVSS

SEQ ID NO: 70 - protein, 110 aa, Artificial Sequence
  Synthetic
  misc_feature (98)..(100): Xaa can be any naturally occurring amino acid
DVVMTQTPSSVSAAVGGTVTIKCQASESIYSDLAWYQQKPGQRPKLLIYFASTLASGVPSRFKGSGSGTEYTLTISGVQREDAATYYCLGDYGNSWAXXXFGAGTKVEIK

SEQ ID NO: 71 - protein, 124 aa, Artificial Sequence
  Synthetic
  misc_feature (3)..(3): Xaa can be any naturally occurring amino acid
  misc_feature (74)..(74): Xaa can be any naturally occurring amino acid
  misc_feature (103)..(113): Xaa can be any naturally occurring amino acid
QSXVEESGGRLVTPGTPLTLTCTVSGIDLSSYSMGWVRQAPGKGLEWIGIIYNTGRIDYANWAKGRFTISKTSXTTVDLKITSPTIGDTATYFCGRGYPGDIXXXXXXXXXXXWGPGTLVTVSS

SEQ ID NO: 72 - protein, 110 aa, Artificial Sequence
  Synthetic
  misc_feature (98)..(100): Xaa can be any naturally occurring amino acid
DGVMTQTPSSTSAAVEGTVTINCQASQSIGSYLSWYQQKPGQPPKLLIYQASKMASGVPSRFKGSGSGTQFTLTISNVQGEDAATYYCLGHYVGDWSXXXFGAGTKVEIK

SEQ ID NO: 73 - protein, 124 aa, Artificial Sequence
  Synthetic
  misc_feature (74)..(74): Xaa can be any naturally occurring amino acid
QEQLKESGGRLVTPGTPLTLTCTASGFSLSTYAMIWVRQAPGKGLEYIGIISYGGSAYYASWAKGRFTISKTSXTTVDLKITSPTTEDTATYFCARDRGFGYGYGIGYYGLDPWGPGTLVTVSS

SEQ ID NO: 74 - protein, 110 aa, Artificial Sequence
  Synthetic
ALVMTQTPPSLSASVGETVRIRCLASEDIYNTISWYQQKPGKPPTLLIYGASNLKSGVPPRFSGSGSGTDYTLTIGGVQSEDAATYYCLGGYSYSSTGLTFGAGTKVEIK

SEQ ID NO: 75 - protein, 124 aa, Artificial Sequence
  Synthetic
  misc_feature (3)..(3): Xaa can be any naturally occurring amino acid
  misc_feature (74)..(74): Xaa can be any naturally occurring amino acid
  misc_feature (113)..(113): Xaa can be any naturally occurring amino acid
QSXVEESGGRLVTPGTPLTLTCTVSGFSLSSYAMTWVRQAPGKGLEWIGVIGTSGMRYYANWAKGRFTISKTSXTTVDLKITSPTTEDTATYFCARANFYVGNDESTDAFDPXWGPGTLVTVSS

SEQ ID NO: 76 - protein, 110 aa, Artificial Sequence
  Synthetic
  misc_feature (99)..(100): Xaa can be any naturally occurring amino acid
DPVLTQTPSSTSAAVGGTVTIKCQASQNIGRWLAWYQQKPGQPPKLLIYRASDLASGVPSRFKGSGSGTQFTLTISGVQREDAATYYCLGSYSSSDSAXXFGGGTEVVVK

SEQ ID NO: 77 - protein, 124 aa, Artificial Sequence
  Synthetic
  misc_feature (3)..(3): Xaa can be any naturally occurring amino acid
  misc_feature (111)..(113): Xaa can be any naturally occurring amino acid
QSXLEESGGRLVTPGTPLTLTCTVSGFSLSTYTLGWVRQAPGKGLEYIGWIDSSGSAYYASWAKGRFTISKASSTTVDLKMAGLTTEDTATYFCASGNTGGDPLYYGMDPXXXWGPGTLVTVSS

SEQ ID NO: 78 - protein, 117 aa, Artificial Sequence
  Synthetic
  misc_feature (35)..(35): Xaa can be any naturally occurring amino acid
  misc_feature (103)..(106): Xaa can be any naturally occurring amino acid
QPVLTQSPSVSAALGASAKLTCTLSSAHKTYSIGXWYQQHQGEAPRFLMQLRRDGSYTKGTGVPDHFSGSSSGADRYLIIPNVQADDEADYYCGVDYDGGYVXXXXFGGGTQLTVTG

SEQ ID NO: 79 - protein, 124 aa, Artificial Sequence
  Synthetic
  misc_feature (3)..(3): Xaa can be any naturally occurring amino acid
  misc_feature (74)..(74): Xaa can be any naturally occurring amino acid
  misc_feature (104)..(113): Xaa can be any naturally occurring amino acid
QSXVEESGGRLVTPGTPLTLTCTVSGFSLSNYIITWVRQAPGKGLEWIGMISTDGDTYYANWAKGRFTISKTSXTTANLKITSPTTEDTATYFCARGLTNFFIXXXXXXXXXXWGPGTLVTVSS

SEQ ID NO: 80 - protein, 117 aa, Artificial Sequence
  Synthetic
  misc_feature (35)..(35): Xaa can be any naturally occurring amino acid
  misc_feature (103)..(106): Xaa can be any naturally occurring amino acid
QPVLTQSPSASATLGSSAKLTCTLSSAHSTYTIAXWYQQKQGESPRCLMYLKSDGSYTRGTGVPDRFSGSSSGADRYLIIPNVQADDEADYYCGADYSGGYLXXXXFGGGTQLTVTG

SEQ ID NO: 81 - protein, 124 aa, Artificial Sequence
  Synthetic
  misc_feature (3)..(3): Xaa can be any naturally occurring amino acid
  misc_feature (74)..(74): Xaa can be any naturally occurring amino acid
  misc_feature (104)..(113): Xaa can be any naturally occurring amino acid
QSXLEESGGGLVTPGTPLTLTCTVSGIDLSAYAMTWVRQAPGKGLEWIGMISTDGATYYASWAKGRFTISKTSXTTADLTITSPTTEDTATYFCARGLTNFFIXXXXXXXXXXWGPGTLVTISS

SEQ ID NO: 82 - protein, 117 aa, Artificial Sequence
  Synthetic
  misc_feature (35)..(35): Xaa can be any naturally occurring amino acid
  misc_feature (103)..(106): Xaa can be any naturally occurring amino acid
QPVLTQSPSASAALGASAKLTCTLSSAHKTYTIAXWYQQQPGESPRYLMLLKSDGSYTRGPGVPDRFSGSSSGADRYLIIPSVQADDEAGYYCGADYNGGYVXXXXFGGGTQLTVTG

SEQ ID NO: 83 - protein, 124 aa, Artificial Sequence
  Synthetic
  misc_feature (3)..(3): Xaa can be any naturally occurring amino acid
  misc_feature (74)..(74): Xaa can be any naturally occurring amino acid
  misc_feature (104)..(113): Xaa can be any naturally occurring amino acid
QSXVEESRGRLVTPGTPLTLTCTVSGIDLSSSAITWVRQAPGKGLEWIGMISTDGATYYANWAKGRFTFSKTSXTTADLKITSPTTEDTATYFCARGLTNFSIXXXXXXXXXXWGPGTLVTVSS

SEQ ID NO: 84 - protein, 117 aa, Artificial Sequence
  Synthetic
  misc_feature (34)..(35): Xaa can be any naturally occurring amino acid
  misc_feature (58)..(61): Xaa can be any naturally occurring amino acid
SFVLTQPASVQVNLGQTVSLTCTADTLSRTYASXXWYQQKPGQAPVLLIYRDTSRPSXXXXGVPDRFSGSSSGNTATLTISGAQAGDEADYYCATSDGSGSSYQYVFGGGTQLTVTG

SEQ ID NO: 85 - protein, 124 aa, Artificial Sequence
  Synthetic
  misc_feature (111)..(113): Xaa can be any naturally occurring amino acid
QEQLVESGGRLVTPGTPLTLTCTVSGFSLSSYHMSWVRQAPGKGLEWIGIIYFTGDASYASWAKGRFTISKTSPTTVDLKITSPTTEDTATYFCARDTDWNSSRTIPFDPXXXWGPGTLVTVSS

SEQ ID NO: 86 - protein, 117 aa, Artificial Sequence
  Synthetic
  misc_feature (58)..(59): Xaa can be any naturally occurring amino acid
  misc_feature (104)..(106): Xaa can be any naturally occurring amino acid
QFVLTQPQSVSGSLGQTVSMSCKCDSGNIDNYYVHWYQQHPGKAPTTVIYNDDQRPSXXGVPDRFSGSIDSTSNSASLTITGLLAEDEADYYCLSSVSSDNYVXXXFGGGTQLTVTG

SEQ ID NO: 87 - protein, 124 aa, Artificial Sequence
  Synthetic
  misc_feature (3)..(3): Xaa can be any naturally occurring amino acid
  misc_feature (74)..(74): Xaa can be any naturally occurring amino acid
  misc_feature (112)..(113): Xaa can be any naturally occurring amino acid
QSXVEESGGRLVTPGTPLTLTCTVSGFSLSSYAMSWVRQAPGKGLEWIGIIYPNDITYYASWAKGRFTISKTSXTTVDLKITSPTTEDTATYFCARVVSWPGYTTEYYFNIXXWGPGTLVTISS

SEQ ID NO: 88 - protein, 23 aa, Artificial Sequence
  Synthetic
DPVLTQTPSSTSAAVGGTVTIKC

SEQ ID NO: 89 - protein, 15 aa, Artificial Sequence
  Synthetic
WYQQKPGQPPKLLIY

SEQ ID NO: 90 - protein, 32 aa, Artificial Sequence
  Synthetic
GVPSRFKGSRSGTEYTLTISGVEREDAATYYC

SEQ ID NO: 91 - protein, 10 aa, Artificial Sequence
  Synthetic
FGGGTKLEIK

SEQ ID NO: 92 - protein, 30 aa, Artificial Sequence
  Synthetic
  misc_feature (16)..(16): Xaa can be any naturally occurring amino acid
QKQLVESGGRLVTPGXSLTLTCTISGIDLN

SEQ ID NO: 93 - protein, 14 aa, Artificial Sequence
  Synthetic
WVRQAPGKGLEYIG

SEQ ID NO: 94 - protein, 31 aa, Artificial Sequence
  Synthetic
  misc_feature (9)..(9): Xaa can be any naturally occurring amino acid
RFTISKTSXTTVDLKMTSLTASDTATYFCAR

SEQ ID NO: 95 - protein, 11 aa, Artificial Sequence
  Synthetic
WGPGTLVTVSS

SEQ ID NO: 96 - protein, 23 aa, Artificial Sequence
  Synthetic
DGVMTQTPSSTSAAVGGTVTIKC

SEQ ID NO: 97 - protein, 15 aa, Artificial Sequence
  Synthetic
WYQQKPGQPPKLLIY

SEQ ID NO: 98 - protein, 32 aa, Artificial Sequence
  Synthetic
GVPSRFKGSRSGTEYTLAISGVQREDAATYYC

SEQ ID NO: 99 - protein, 10 aa, Artificial Sequence
  Synthetic
FGAGTNVEIK

SEQ ID NO: 100 - protein, 30 aa, Artificial Sequence
  Synthetic
  misc_feature (3)..(3): Xaa can be any naturally occurring amino acid
QSXLEESGGRLVTPGTPLTLTCTVSGFSLS

SEQ ID NO: 101 - protein, 14 aa, Artificial Sequence
  Synthetic
WVRQAPGKGLEYIG

SEQ ID NO: 102 - protein, 31 aa, Artificial Sequence
  Synthetic
RFTISKTSSTTVDLKMTSLTTEDTATYFCAS

SEQ ID NO: 103 - protein, 11 aa, Artificial Sequence
  Synthetic
WGPGTLVTVSS

SEQ ID NO: 104 - protein, 23 aa, Artificial Sequence
  Synthetic
DVVMTQTPSSVSAAVGGTVTIKC

SEQ ID NO: 105 - protein, 15 aa, Artificial Sequence
  Synthetic
WYQQKPGQRPKLLIY

SEQ ID NO: 106 - protein, 32 aa, Artificial Sequence
  Synthetic
GVPSRFKGSGSGTEYTLTISGVQREDAATYYC

SEQ ID NO: 107 - protein, 10 aa, Artificial Sequence
  Synthetic
FGAGTKVEIK

SEQ ID NO: 108 - protein, 30 aa, Artificial Sequence
  Synthetic
  misc_feature (3)..(3): Xaa can be any naturally occurring amino acid
QSXVEESGGRLVTPGTPLTLTCTVSGIDLS

SEQ ID NO: 109 - protein, 14 aa, Artificial Sequence
  Synthetic
WVRQAPGKGLEWIG

SEQ ID NO: 110 - protein, 31 aa, Artificial Sequence
  Synthetic
  misc_feature (9)..(9): Xaa can be any naturally occurring amino acid
RFTISKTSXTTVDLKITSPTIGDTATYFCGR

SEQ ID NO: 111 - protein, 11 aa, Artificial Sequence
  Synthetic
WGPGTLVTVSS

SEQ ID NO: 112 - protein, 23 aa, Artificial Sequence
  Synthetic
DGVMTQTPSSTSAAVEGTVTINC

SEQ ID NO: 113 - protein, 15 aa, Artificial Sequence
  Synthetic
WYQQKPGQPPKLLIY

SEQ ID NO: 114 - protein, 32 aa, Artificial Sequence
  Synthetic
GVPSRFKGSGSGTQFTLTISNVQGEDAATYYC

SEQ ID NO: 115 - protein, 10 aa, Artificial Sequence
  Synthetic
FGAGTKVEIK

SEQ ID NO: 116 - protein, 30 aa, Artificial Sequence
  Synthetic
QEQLKESGGRLVTPGTPLTLTCTASGFSLS

SEQ ID NO: 117 - protein, 14 aa, Artificial Sequence
  Synthetic
WVRQAPGKGLEYIG

SEQ ID NO: 118 - protein, 31 aa, Artificial Sequence
  Synthetic
  misc_feature (9)..(9): Xaa can be any naturally occurring amino acid
RFTISKTSXTTVDLKITSPTTEDTATYFCAR

SEQ ID NO: 119 - protein, 11 aa, Artificial Sequence
  Synthetic
WGPGTLVTVSS

SEQ ID NO: 120 - protein, 23 aa, Artificial Sequence
  Synthetic
ALVMTQTPPSLSASVGETVRIRC

SEQ ID NO: 121 - protein, 15 aa, Artificial Sequence
  Synthetic
WYQQKPGKPPTLLIY

SEQ ID NO: 122 - protein, 32 aa, Artificial Sequence
  Synthetic
GVPPRFSGSGSGTDYTLTIGGVQSEDAATYYC

SEQ ID NO: 123 - protein, 10 aa, Artificial Sequence
  Synthetic
FGAGTKVEIK

SEQ ID NO: 124 - protein, 30 aa, Artificial Sequence
  Synthetic
  misc_feature (3)..(3): Xaa can be any naturally occurring amino acid
QSXVEESGGRLVTPGTPLTLTCTVSGFSLS

SEQ ID NO: 125 - protein, 14 aa, Artificial Sequence
  Synthetic
WVRQAPGKGLEWIG

SEQ ID NO: 126 - protein, 31 aa, Artificial Sequence; identical to SEQ ID NO: 118
  Synthetic
  misc_feature (9)..(9): Xaa can be any naturally occurring amino acid

SEQ ID NO: 127 - protein, 11 aa, Artificial Sequence
  Synthetic
WGPGTLVTVSS

SEQ ID NO: 128 - protein, 23 aa, Artificial Sequence
  Synthetic
DPVLTQTPSSTSAAVGGTVTIKC

SEQ ID NO: 129 - protein, 15 aa, Artificial Sequence
  Synthetic
WYQQKPGQPPKLLIY

SEQ ID NO: 130 - protein, 32 aa, Artificial Sequence
  Synthetic
GVPSRFKGSGSGTQFTLTISGVQREDAATYYC

SEQ ID NO: 131 - protein, 10 aa, Artificial Sequence
  Synthetic
FGGGTEVVVK

SEQ ID NO: 132 - protein, 30 aa, Artificial Sequence; identical to SEQ ID NO: 100
  Synthetic
  misc_feature (3)..(3): Xaa can be any naturally occurring amino acid

SEQ ID NO: 133 - protein, 14 aa, Artificial Sequence
  Synthetic
WVRQAPGKGLEYIG

SEQ ID NO: 134 - protein, 31 aa, Artificial Sequence
  Synthetic
RFTISKASSTTVDLKMAGLTTEDTATYFCAS

SEQ ID NO: 135 - protein, 11 aa, Artificial Sequence
  Synthetic
WGPGTLVTVSS

SEQ ID NO: 136 - protein, 22 aa, Artificial Sequence
  Synthetic
QPVLTQSPSVSAALGASAKLTC

SEQ ID NO: 137 - protein, 15 aa, Artificial Sequence
  Synthetic
WYQQHQGEAPRFLMQ

SEQ ID NO: 138 - protein, 34 aa, Artificial Sequence
  Synthetic
GTGVPDHFSGSSSGADRYLIIPNVQADDEADYYC

SEQ ID NO: 139 - protein, 11 aa, Artificial Sequence
  Synthetic
FGGGTQLTVTG

SEQ ID NO: 140 - protein, 30 aa, Artificial Sequence; identical to SEQ ID NO: 124
  Synthetic
  misc_feature (3)..(3): Xaa can be any naturally occurring amino acid

SEQ ID NO: 141 - protein, 14 aa, Artificial Sequence
  Synthetic
WVRQAPGKGLEWIG

SEQ ID NO: 142 - protein, 31 aa, Artificial Sequence
  Synthetic
  misc_feature (9)..(9): Xaa can be any naturally occurring amino acid
RFTISKTSXTTANLKITSPTTEDTATYFCAR

SEQ ID NO: 143 - protein, 11 aa, Artificial Sequence
  Synthetic
WGPGTLVTVSS

SEQ ID NO: 144 - protein, 22 aa, Artificial Sequence
  Synthetic
QPVLTQSPSASATLGSSAKLTC

SEQ ID NO: 145 - protein, 15 aa, Artificial Sequence
  Synthetic
WYQQKQGESPRCLMY

SEQ ID NO: 146 - protein, 34 aa, Artificial Sequence
  Synthetic
GTGVPDRFSGSSSGADRYLIIPNVQADDEADYYC

SEQ ID NO: 147 - protein, 11 aa, Artificial Sequence
  Synthetic
FGGGTQLTVTG

SEQ ID NO: 148 - protein, 30 aa, Artificial Sequence
  Synthetic
  misc_feature (3)..(3): Xaa can be any naturally occurring amino acid
QSXLEESGGGLVTPGTPLTLTCTVSGIDLS

SEQ ID NO: 149 - protein, 14 aa, Artificial Sequence
  Synthetic
WVRQAPGKGLEWIG

SEQ ID NO: 150 - protein, 31 aa, Artificial Sequence
  Synthetic
  misc_feature (9)..(9): Xaa can be any naturally occurring amino acid
RFTISKTSXTTADLTITSPTTEDTATYFCAR

SEQ ID NO: 151 - protein, 11 aa, Artificial Sequence
  Synthetic
WGPGTLVTISS

SEQ ID NO: 152 - protein, 22 aa, Artificial Sequence
  Synthetic
QPVLTQSPSASAALGASAKLTC

SEQ ID NO: 153 - protein, 15 aa, Artificial Sequence
  Synthetic
WYQQQPGESPRYLML

SEQ ID NO: 154 - protein, 34 aa, Artificial Sequence
  Synthetic
GPGVPDRFSGSSSGADRYLIIPSVQADDEAGYYC

SEQ ID NO: 155 - protein, 11 aa, Artificial Sequence
  Synthetic
FGGGTQLTVTG

SEQ ID NO: 156 - protein, 30 aa, Artificial Sequence
  Synthetic
  misc_feature (3)..(3): Xaa can be any naturally occurring amino acid
QSXVEESRGRLVTPGTPLTLTCTVSGIDLS

SEQ ID NO: 157 - protein, 14 aa, Artificial Sequence
  Synthetic
WVRQAPGKGLEWIG

SEQ ID NO: 158 - protein, 31 aa, Artificial Sequence
  Synthetic
  misc_feature (9)..(9): Xaa can be any naturally occurring amino acid
RFTFSKTSXTTADLKITSPTTEDTATYFCAR

SEQ ID NO: 159 - protein, 11 aa, Artificial Sequence
  Synthetic
WGPGTLVTVSS

SEQ ID NO: 160 - protein, 22 aa, Artificial Sequence
  Synthetic
SFVLTQPASVQVNLGQTVSLTC

SEQ ID NO: 161 - protein, 15 aa, Artificial Sequence
  Synthetic
WYQQKPGQAPVLLIY

SEQ ID NO: 162 - protein, 34 aa, Artificial Sequence
  Synthetic
  misc_feature (1)..(2): Xaa can be any naturally occurring amino acid
XXGVPDRFSGSSSGNTATLTISGAQAGDEADYYC

SEQ ID NO: 163 - protein, 11 aa, Artificial Sequence
  Synthetic
FGGGTQLTVTG

SEQ ID NO: 164 - protein, 30 aa, Artificial Sequence
  Synthetic
QEQLVESGGRLVTPGTPLTLTCTVSGFSLS

SEQ ID NO: 165 - protein, 14 aa, Artificial Sequence
  Synthetic
WVRQAPGKGLEWIG

SEQ ID NO: 166 - protein, 31 aa, Artificial Sequence
  Synthetic
RFTISKTSPTTVDLKITSPTTEDTATYFCAR

SEQ ID NO: 167 - protein, 11 aa, Artificial Sequence
  Synthetic
WGPGTLVTVSS

SEQ ID NO: 168 - protein, 22 aa, Artificial Sequence
  Synthetic
QFVLTQPQSVSGSLGQTVSMSC

SEQ ID NO: 169 - protein, 15 aa, Artificial Sequence
  Synthetic
WYQQHPGKAPTTVIY

SEQ ID NO: 170 - protein, 34 aa, Artificial Sequence
  Synthetic
GVPDRFSGSIDSTSNSASLTITGLLAEDEADYYC

SEQ ID NO: 171 - protein, 11 aa, Artificial Sequence
  Synthetic
FGGGTQLTVTG

SEQ ID NO: 172 - protein, 30 aa, Artificial Sequence; identical to SEQ ID NO: 124
  Synthetic
  misc_feature (3)..(3): Xaa can be any naturally occurring amino acid

SEQ ID NO: 173 - protein, 14 aa, Artificial Sequence
  Synthetic
WVRQAPGKGLEWIG

SEQ ID NO: 174 - protein, 31 aa, Artificial Sequence; identical to SEQ ID NO: 118
  Synthetic
  misc_feature (9)..(9): Xaa can be any naturally occurring amino acid

SEQ ID NO: 175 - protein, 11 aa, Artificial Sequence
  Synthetic
WGPGTLVTISS

SEQ ID NO: 176 - protein, 20 aa, Artificial Sequence
  Synthetic
PDGVFQEDAPVIHQEMIGGL

SEQ ID NO: 177 - protein, 33 aa, Artificial Sequence
  Synthetic
KDICEEQVNSLPNSLPGSITKAGDFLEDFLEAN

SEQ ID NO: 178 - protein, 16 aa, Artificial Sequence
  Synthetic
KDAPDHQELNLDVSLQ

SEQ ID NO: 179 - protein, 11 aa, Artificial Sequence
  Synthetic
  MISC_FEATURE (1)..(1): X can be Gln, Leu
  MISC_FEATURE (4)..(4): X can be Gln, Glu
  MISC_FEATURE (5)..(5): X can be Asn, Ser, Asp
  MISC_FEATURE (7)..(7): X can be Tyr, Arg, Gly
  MISC_FEATURE (8)..(8): X can be Thr, Ser, Asn, Arg
  MISC_FEATURE (9)..(9): X can be Ser, Trp, Asp, Tyr, Thr
  MISC_FEATURE (10)..(10): X can be Leu, Ile
  MISC_FEATURE (11)..(11): X can be Ala, Ser
XASXXIXXXXX

SEQ ID NO: 180 - protein, 7 aa, Artificial Sequence
  Synthetic
  MISC_FEATURE (1)..(1): X can be Gly, Glu, Phe, Gln, Arg
  MISC_FEATURE (4)..(4): X can be Asn, Lys, Thr, Asp
  MISC_FEATURE (5)..(5): X can be Leu, Met
  MISC_FEATURE (6)..(6): X can be Ala, Lys
XASXXXS

SEQ ID NO: 181 - protein, 14 aa, Artificial Sequence
  Synthetic
  MISC_FEATURE (3)..(3): X can be Ser, Asp, His, Gly
  MISC_FEATURE (4)..(4): X can be Tyr, Ile
  MISC_FEATURE (5)..(5): X can be Thr, Ala, Gly, Val, Ser
  MISC_FEATURE (6)..(6): X can be  Gln, Ser, Asn, Gly, Tyr
  MISC_FEATURE (7)..(7): X can be Ile, Asp, Ser
  MISC_FEATURE (8)..(8): X can be Asp, Trp, Ser
  MISC_FEATURE (9)..(9): X can be Thr, Ala, Ser
  MISC_FEATURE (10)..(12): X can be any amino acid
  misc_feature (13)..(14): Xaa can be any naturally occurring amino acid
LGXXXXXXXXXXXX

SEQ ID NO: 182 - protein, 13 aa, Artificial Sequence
  Synthetic
  MISC_FEATURE (1)..(1): X can be Thr, Lys
  MISC_FEATURE (2)..(2): X can be Leu, Ala, Cys
  MISC_FEATURE (3)..(3): X can be Ser, Asp
  MISC_FEATURE (4)..(4): X can be Ser, Thr
  MISC_FEATURE (5)..(5): X can be Ala, Leu, Gly
  MISC_FEATURE (6)..(6): X can be His, Ser, Asn
  MISC_FEATURE (7)..(7): X can be Lys, Ser, Arg, Ile
  MISC_FEATURE (8)..(8): X can be Thr, Asp
  MISC_FEATURE (9)..(9): X can be Tyr, Asn
  MISC_FEATURE (10)..(10): X can be Ser, Thr, Ala, Tyr
  MISC_FEATURE (11)..(11): X can be Ile, Ser, Tyr
  MISC_FEATURE (12)..(13): X can be any amino acid
XXXXXXXXXXXXX

SEQ ID NO: 183 - protein, 9 aa, Artificial Sequence
  Synthetic
  MISC_FEATURE (1)..(1): X can be Leu, Arg, Asn
  MISC_FEATURE (2)..(2): X can be Arg, Lys, Asp
  MISC_FEATURE (3)..(3): X can be Arg, Ser, Thr, Asp
  MISC_FEATURE (4)..(4): X can be Asp, Ser, Gln
  MISC_FEATURE (5)..(5): X can be Gly, Arg
  MISC_FEATURE (6)..(6): X can be Ser, Pro
  MISC_FEATURE (7)..(7): X can be Tyr, Ser
  MISC_FEATURE (8)..(9): X can be any amino acid
XXXXXXXXX

SEQ ID NO: 184 - protein, 13 aa, Artificial Sequence
  Synthetic
  MISC_FEATURE (1)..(1): X can be Gly, Ala, Leu
  MISC_FEATURE (2)..(2): X can be Val, Ala, Thr, Ser
  MISC_FEATURE (3)..(3): X can be Asp, Ser
  MISC_FEATURE (4)..(4): X can be Tyr, Asp, Val
  MISC_FEATURE (5)..(5): X can be Asp, Ser, Asn, Gly
  MISC_FEATURE (6)..(6): X can be Gly, Ser
  MISC_FEATURE (7)..(7): X can be Gly, Asp
  MISC_FEATURE (8)..(8): X can be Tyr, Ser, Asn
  MISC_FEATURE (9)..(9): X can be Val, Leu, Ser, Tyr
  MISC_FEATURE (10)..(13): X can be any amino acid
XXXXXXXXXXXXX

SEQ ID NO: 185 - protein, 5 aa, Artificial Sequence
  Synthetic
  MISC_FEATURE (1)..(1): X can be Thr, Arg, Asn, Ser, Ala
  MISC_FEATURE (2)..(2): X can be Tyr, Ser
  MISC_FEATURE (3)..(3): X can be Gly, Thr, Ile, Ser, Ala, His, Thr
  MISC_FEATURE (4)..(4): X can be Met, Ile, Leu
  MISC_FEATURE (5)..(5): X can be Thr, Ala, Gly, Ile, Ser
XXXXX

SEQ ID NO: 186 - protein, 16 aa, Artificial Sequence
  Synthetic
  MISC_FEATURE (1)..(1): X can be Ile, Trp, Met, Val
  MISC_FEATURE (3)..(3): X can be Val, Asp, Ser, Tyr, Gly
  MISC_FEATURE (4)..(4): X can be Ser, Thr, Asn, Tyr, Phe, Pro
  MISC_FEATURE (5)..(5): X can be Ser, Asp, Thr, Gly, Asn
  MISC_FEATURE (6)..(6): X can be Gly, Asp
  MISC_FEATURE (7)..(7): X can be Asn, Ser, Asp, Arg, Ala, Met, Ile
  MISC_FEATURE (8)..(8): X can be Thr, Ile, Ala, Arg
  MISC_FEATURE (9)..(9): X can be Tyr, Asp, Ser
  MISC_FEATURE (12)..(12): X can be Asn, Ser
  MISC_FEATURE (15)..(15): X can be Gln, Lys
XIXXXXXXXYAXWAXG

SEQ ID NO: 187 - protein, 17 aa, Artificial Sequence
  Synthetic
  MISC_FEATURE (1)..(1): X can be Gly, Asp, Ala, Val
  MISC_FEATURE (2)..(2): X can be Leu, Asn, Tyr, Arg, Thr, Val
  MISC_FEATURE (3)..(3): X can be Gly, Asp, Thr, Pro, Phe, Ser
  MISC_FEATURE (4)..(4): X can be Arg, Gly, Asn, Phe, Trp, Tyr
  MISC_FEATURE (5)..(5): X can be Ala, Gly, Phe, Asp, Asn, Val, Pro
  MISC_FEATURE (6)..(6): X can be Tyr, Asp, Phe, Ile, Ser, Gly
  MISC_FEATURE (7)..(17): X can be any amino acid
XXXXXXXXXXXXXXXXX

SEQ ID NO: 188 - protein, 17 aa, Artificial Sequence
  Synthetic
KDAPDHQELNLDVSLQL

SEQ ID NO: 189 - protein, 17 aa, Artificial Sequence
  Synthetic
KDVPDHKELNLDVSLQL

SEQ ID NO: 190 - protein, 17 aa, Artificial Sequence
  Synthetic
KDVPNHKDLNLQVSINL

SEQ ID NO: 191 - protein, 17 aa, Artificial Sequence
  Synthetic
TDVPDHKDLNMDVSFHL

SEQ ID NO: 192 - protein, 13 aa, Artificial Sequence
  Synthetic
LQEAKDICEEQVN

SEQ ID NO: 193 - protein, 7 aa, Artificial Sequence
  Synthetic
QYQKDAP

SEQ ID NO: 194 - DNA, 24 bp, Artificial Sequence
  synthetic
  misc_feature (10)..(10): wherein N is t or c
gaagactganggagccttaggttg

(19) United States
(12) Patent Application Publication
(10) Pub. No.: US 2023/0029635 A1
(43) Pub. Date: Feb. 2, 2023

(54) ANTIBODIES TARGETING CELL SURFACE DEPOSITED COMPLEMENT PROTEIN C3d AND USE THEREOF

(71) Applicants: The United States of America,as represented by the Secretary,Department of Health and Human Services, Bethesda, MD (US); The Scripps Research Institute, La Jolla, CA (US)

(72) Inventors: Adrian Wiestner, Bethesda, MD (US); Christoph Rader, Jupiter, FL (US); Haiyong Peng, Jupiter, FL (US); Sivasubramanian Baskar, Ellicott City, MD (US); Erika Gaglione, Germantown, MD (US)

(73) Assignees: The United States of America,as represented by the Secretary,Department of Health and Human Services, Bethesda, MD (US); The Scripps Research Institute, La Jolla, CA (US)

(21) Appl. No.: 17/783,345

(22) PCT Filed: Dec. 8, 2020
(86) PCT No.: PCT/US2020/063809

§ 371 Date: Jun. 8, 2022

Related U.S. Application Data
(60) Provisional application No. 62/945,569, filed on Dec. 9, 2019.

(51) Int. Cl. C07K 16/28; A61P 35/00

(52) U.S. Cl. CPC C07K 16/2896; A61P 35/00; A61K 2039/505

(57) ABSTRACT

Disclosed are anti-C3d antibodies or fragments thereof. Also disclosed are methods of killing cancer cells, methods of preparing anti-C3d antibodies, and pharmaceutical compositions.

### CROSS-REFERENCE TO RELATED APPLICATION

This patent application claims the benefit of co-pending U.S. Provisional Patent Application No. 62/945,569, filed Dec. 9, 2019, which is incorporated by reference in its entirety herein.

### STATEMENT REGARDING FEDERALLY SPONSORED RESEARCH OR DEVELOPMENT

This invention was made with Government support under project number ZIAHL006070-09 by the National Institutes of Health, National Cancer Institute. The Government has certain rights in this invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 82,529 Byte ASCII (Text) file named “750952_ST25.txt,” created on Dec. 8, 2020.

### BACKGROUND OF THE INVENTION

Monoclonal antibodies (mAbs) have become a mainstay of therapy for many cancers. The key effector mechanisms of mAbs are induction of cell death through complement-dependent cytotoxicity (CDC), antibody-dependent cellular cytotoxicity (ADCC), antibody-dependent cell-mediated phagocytosis (ADCMP) and in some cases may include induction of apoptosis. The most commonly used monoclonal antibodies are of mouse origin that have been chimerized or humanized to carry human constant regions (typically the human lgG1 isotype), a requirement for the recruitment of human effector mechanisms.

However, antibody therapy is not completely effective in some applications due to loss of the target surface antigen. For instance, rituximab and ofatumumab are anti-CD20 monoclonal antibodies that mediate human immune effector mechanisms including CDC as well as ADCC and ADCMP and are approved for patients with Chronic Lymphocytic Leukemia (CLL), a B-cell malignancy. Upon infusion of either of these antibodies, complement protein is deposited on the cell surface of CLL cells and a subset of the cells is killed; however, other CLL cells escape, having lost CD20 expression due to a process called trogocytosis by which antibody-CD20 complexes are pulled off the CLL cell surface by immune cells that bind the Fc-portion of the antibody. The process of trogocytosis leading to antigen loss is not limited to anti-CD20 antibodies or leukemia and lymphoma but appears to be a common event in antibody therapy.

Antibody C8xi, disclosed in U.S. Pat. No. 10,035,848, is an effective C3d antibody that binds to the human epitope KDAPDHQELNLDVSLQL (SEQ ID NO: 188). As seen in the sequences below, this epitope is not conserved in macaque, dog, and mouse (bold indicates variation). This lack of conservation may limit the use of C8xi in some animal disease models.

Human:
(SEQ ID NO: 188)
KDAPDHQELNLDVSLQL
Macaque:
(SEQ ID NO: 189
KDVPDHKELNLDVSLQL
Dog:
(SEQ ID NO: 190)
KDVPNHKDLNLQVSINL
Mouse:
(SEQ ID NO: 191)
TDVPDHKDLNMDVSFHL

While antibodies, such as C8xi, have been developed that successfully overcome problems associated with loss of the targeted antigen (e.g., U.S. Pat. No. 10,035,848), there is still a need for new antibody therapies that can overcome problems associated with loss of the targeted antigen and can bind to a human epitope that is conserved in other animals.

### BRIEF SUMMARY OF THE INVENTION

Provided is an antibody or antibody fragment immunospecific for human complement protein C3d. According to one aspect of the invention, the anti-C3d antibody or antibody fragment comprises (a) SEQ ID NOs: 179-181 or 182-184; and (b) SEQ ID NOs: 185-187; or a sequence with at least about 97% sequence identity thereto.

The invention also provides a method of using the antibody or antibody fragment to kill cells having C3d deposited on their surface.

Further provided is a polypeptide comprising (a) SEQ ID NO: 192 or SEQ ID NO: 193 (b) a fragment of five or more contiguous amino acids of SEQ ID NO: 192 or SEQ ID NO: 193; or (c) a combination thereof; wherein the polypeptide has fewer than 50 total amino acids and the polypeptide inhibits binding of an antibody or antibody fragment of claim 1 to C3d.

Related compositions, cells, nucleic acids, and methods also are provided as is apparent from the following detailed description of the invention.

### BRIEF DESCRIPTION OF THE SEVERAL VIEWS OF THE DRAWING(S)

FIG. 1 is a graph showing the binding of antibodies of the present invention to immobilized human C3d in the absence (PBS) or presence of normal human serum (NHS) at 1/2, 1/5, 1/10, 1/20, and 1/50 dilutions. A loss of signal in the presence of NHS indicates competition by full-length C3 protein, an abundant serum protein. The binding of 3NS1 and 5S8 to immobilized human C3d was not or only minimally reduced, respectively, in the presence of NHS. Each bank of bars in the bar graph represents, from left to right, a PBS control and the indicated antibody tested at dilutions of 1/2, 1/5, 1/10, 1/20, and 1/50.

FIG. 2 is a graph showing the ability of 5S8 and d301 antibodies of the present invention to induce antibody-dependent cellular phagocytosis (ADCP) of primary CLL cells. “Pre-OFA” refers to patients with chronic lymphocytic leukemia before treatment with anti-CD20 antibodies and “24 hr Post-OFA” refers to patients 24 hours after ofatumumab (an anti-CD20 antibody) administration. “OFA” refers to ofatumumab and “RTX” refers to rituximab.

FIG. 3 is a graph showing that administration of anti-C3d mAbs 5S8 and d301 of the present invention in combination with ofatumumab increases survival of lymphoma carrying mice in the HBL-2 model (Mantle Cell Lymphoma cell line). BALB/c scid mice were challenged s.c. with 5 million HBL-2 cells and were randomized on day 7 (five per group) to different treatments. The survival probability is plotted against days after tumor challenge. “OFA” refers to ofatumumab.

FIG. 4 is a graph showing that administration of the d301 mAb of the present invention in combination with ofatumumab increases survival of lymphoma carrying mice in the HBL-2 model (Mantle Cell Lymphoma cell line). BALB/c scid mice were challenged s.c. with 5 million HBL-2 cells and were randomized on day 7 (five per group) to different treatments. The survival probability is plotted against days after tumor challenge. “OFA” refers to ofatumumab and “TRA” refers to trastuzumab. TRA is included as a non-targeting control mAb, i.e. trastuzumab has no binding sites on the tumor cells.

FIG. 5 is a graph showing combined data from the HBL-2 model (Mantle Cell Lymphoma cell line) as shown for individual experiments in FIGS. 3 and 4. Administration of anti-C3d mAb (5S8 or d301) of the present invention in combination with ofatumumab significantly increases survival of lymphoma carrying mice. Single agent ofatumumab or anti-C3d mAb in combination with a non-targeting control mAb (trastuzumab, TRA) slightly extends survival but all mice succumb to disease. With combination therapy 8 of 15 (53%) of mice survived long-term. Balb/c-SCID mice were challenged s.c. with 5 million HBL-2 cells and were randomized on day 7 to no treatment (no Ab, n=6) or four weekly i.p. injections of mAbs: 20 mg/kg ofatumumab alone (OFA, n=9) or two mAbs combined, each dosed at 10 mg/kg for total dose of 20 mg/kg: trastuzumab (TRA)+anti-C3d mAb (d301, n=5), OFA+anti-C3d mAb (n=15; in 7 mice 5S8; in 8 mice d301). OFA+aC3d vs OFA, p=0.01; OFA+aC3d vs TRA+aC3d, p=0.006.

FIGS. 6A and 6B are graphs showing that administration of anti-C3d antibodies of the present invention in combination with ofatumumab significantly increases mouse survival in the SUDHL-6 model (aggressive lymphoma of the Diffuse Large B cell Lymphoma type) (panel A). Balb/c-SCID mice were challenged s.c. with 5e6 SUDHL-6 cells and were randomized (n=5 per group) when tumors became palpable in most mice (day 7). The time palpable tumors were noted is indicated by “P” in panel B. The cohorts received either no treatment or weekly i.p. injections of 10 mg/kg OFA mixed with 10 mg/kg trastuzumab (TRA, non-targeting control), or an anti-C3d antibody, either 5S8 or d301. Total of eight weekly injections starting on day 7 were given.

FIGS. 7A and 7B are graphs showing that administration of anti-C3d antibodies of the present invention in combination with rituximab significantly increases mouse survival in the SUDHL-6 model (aggressive lymphoma of the Diffuse Large B cell Lymphoma type) (panel A). Balb/c-SCID mice were challenged s.c. with 5 million SUDHL-6 cells and were randomized (n=5 per group) when tumors became palpable in most mice (day 7). The time palpable tumors were noted is indicated by “P” in panel B. The cohorts received either no treatment or weekly i.p. injections of 10 mg/kg RTX mixed with 10 mg/kg trastuzumab (TRA, non-targeting control), or an anti-C3d antibody, either 5S8 or d301. Total of eight weekly injections starting on day 7 were given.

FIG. 8 is a graph showing combined data from the SUDHL-6 model (aggressive lymphoma of the Diffuse Large B cell Lymphoma type) as shown for individual experiments in FIGS. 6A-6B and 7A-7B. Administration of anti-C3d mAb (5S8 or d301) of the present invention in combination with an anti-CD20 antibody (ofatumumab or rituximab) significantly increases survival of lymphoma carrying mice. Therapy with anti-CD20 mAbs improved survival over untreated animals (P<0.0001). The addition of an anti-C3d mAb to the anti-CD20 mAb improved survival further achieving long-term disease-free survival of 75% for combination therapy vs 20% for single agent anti-CD20 (P=0.008).

FIG. 9 is a graph showing that administration of anti-C3d antibodies of the present invention in combination with rituximab could increase the cure rate of Balb/c-SCID mice challenged with SUDHL-4 cells s.c. compared to mice treated with rituximab alone (not statistically significant). Twenty million SUDHL-4cells (aggressive lymphoma of the diffuse large B cell lymphoma type) were injected into Balb/c-SCID mice and were randomized to treatment (n=5 per group). The cohorts received either no mAb (untreated) or weekly i.p. injections of 20 mg/kg RTX alone or 10 mg/kg RTX mixed with 10 mg/kg 5S8 or d301. The weekly injection of mAbs was started on day 7 and continued until day 56. Surviving mice were sacrificed on Day 175 and found to be tumor free. Blood and tissue was used to assess for evidence of complications from treatment (Table 4).

FIG. 10 is a set of flow cytometry plots showing the binding activity of antibodies of the invention to native C3 fragments of different species. Human, or cynomolgus, or mouse C3b/iC3b/C3d were deposited on zymosan particles after incubation with 50% corresponding serum at 37° C. for 45 min and detected by 5 μg/mL Fabs with a 6xHis tag, followed by a secondary mouse anti-6xHIS mAb conjugated to Alexa Fluor 488.

FIGS. 11A-1B are graphs showing the level of competition binding of antibodies of the invention and C8xi to human C3 fragments deposited on zymosan and C3 in human serum. The data in FIG. 8A is from an assay wherein zymosan particles were deposited with human C3 fragments and premixed with different amounts of human serum prior to addition of Fab with 6xHis tag at 1 μg/mL. The data in FIG. 8B is from an assay wherein the antibodies of the invention and C8xi with the 6xHis tag at 15 μg/mL were premixed with different amounts of human serum before adding to zymosan particles deposited with human C3 fragments. Mouse anti-6xHIS mAb conjugated with HRP was used to determine the binding signal of each Fab. Each bank of bars in FIG. 8A represents, from left to right, a PBS control and the indicated antibody tested at dilutions of 1/10, 1/50, 1/1250, 1/1250, and 1/6250. Each bank of bars in FIG. 8B represents, from left to right, a PBS control and the indicated antibody tested at dilutions of 1/2, 1/5, 1/10, 1/20, 1/50, 1/250, and 1/1250.

FIG. 12 is a graph showing the level of competition binding of chimeric rabbit/human Fabs with chimeric mouse/human IgG C8D6 to native human C3 fragments. Each Fab with a 6xHis tag at 1 μg/mL was premixed with different concentrations of chimeric mouse/human anti-human C3d mAb C8xi before adding to zymosan particles deposited with human C3 fragments. Mouse anti-6xHIS mAb conjugated with HRP was used to distinguish the binding signal of Fabs from C8D6 IgG1. Each bank of bars in the bar graph represents, from left to right, a PBS control and the indicated antibody tested at dilutions of 10, 2, 0.4, 0.08 and 0.016 μg/mL.

FIG. 13 is a graph showing the binding activity of antibodies of the invention to different human C3 fragments. The binding of each chimeric rabbit/human Fab at 2.5 μg/mL to C3b/iC3b/(C3d), or C3d was determined by flow cytometry against JeKo-1 cells deposited with human C3 fragments after opsonization by 10 μg/mL rituximab (RTX) followed by incubation with 50% CS-depleted human serum for 2 h with or without 0.5 U/mL CR1 or 24 h. In the presence of CR1 or after treatment with serum for 24 h, most of C3 fragments had decayed from C3b/iC3b to C3d.

FIG. 14 is a graph showing epitope binning of selected anti C3d Fabs by SPR. hFc-hC3d was captured on the chip (RU=38) and then 500 nM Fabs were injected sequentially to identify independent and overlapping epitopes. Resonance unit (RU, y axis) increases that exceeded the values found for previously injected Fabs indicated independent epitopes because they allow simultaneous binding. For example, the increase found for the binding of Fab 5S8 exceeded the values found for C8D3 alone, indicating that Fab 5S8 and C8xi can bind simultaneously to hC3d. By contrast, the epitope of Fab 3N07 overlaps with the epitopes of Fab d301.

### DETAILED DESCRIPTION OF THE INVENTION

Provided is an antibody or antibody fragment which can enhance efficacy of antibody therapy for cancer. Specifically, provided is an antibody or antibody fragment immunospecific for complement protein C3d, and a method of using the antibody or antibody fragment to kill cells having C3d deposited on the surface thereof. C3d is a protein of the complement system. The complement system consists of soluble plasma proteins and is activated upon binding of a mAb to target cells, resulting in the deposition of complement components on the cell surface and formation of the membrane attack complex (MAC), which can kill cells by forming holes in the cell membrane (lysis). The most abundant complement protein is C3. Upon complement activation, C3 is attracted to the cell surface and activated in a proteolytic step, and the product, activated C3b, is deposited on the cell surface followed later by its proteolytic processing to inactive forms, iC3b, C3dg and finally C3d. C3dg and C3d are the final products that remain deposited on the cell membrane for days to weeks, while C3b and iC3b are intermediate products that are further processed within hours. C3d, therefore, provides a stable antigenic target. Without wishing to be bound by any particular theory or mechanism of action, it is believed that the antibodies of the invention bind C3d on the surface of a target cell and, thereby, target the cell for destruction by the host's immune system effector cells (e.g., monocytes, macrophages, NK cells, and neutrophils).

The anti-C3d antibody or antibody fragment is immunospecific for human C3d complement protein, particularly human C3d complement protein on the surface of an opsonized cancer cell, and/or the C3d precursor protein C3dg, which has a very similar amino acid sequence. In some embodiments, the anti-C3d antibody or antibody fragment has a binding affinity (K_{d}) for human C3d protein of at least 500 nM. Desirably, the anti-C3d antibody or antibody fragment has an affinity for C3d that is sufficiently greater than its affinity for other complement proteins that it does not cross react with other complement proteins, particularly C3, which might otherwise compete with C3d for antibody binding, with the exception that the antibody or antibody fragment may cross-react with C3dg or C3b/iC3b. In some embodiments, the anti-C3d antibody binds an epitope on C3d comprising, or falling within, any of the amino acid sequences of SEQ ID NO: 192 and 193.

Further provided is an antibody or antibody fragment that binds to epitopes of C3d that are conserved in other animals, such as non-human primates, dogs, and mice, which allows for the effective use of the antibodies in pre-clinical models (Table 2).

The anti-C3d antibody or antibody fragment comprises a variable region that contains complementary determining regions (CDRs), which determine the binding specificity of the antibody or antibody fragment. The variable region may include heavy and light chains each comprising CDR regions (wherein the CDRs of the light chain can be referred to as CDRL1, CDRL2, and CDRL3, and the CDRs of the heavy chain can be referred to as CDRH1, CDRH2, and CDRH3.

In an embodiment, the antibody or antibody fragment comprises: (a) CDRL1, CDRL2, and CDRL3 of SEQ ID NOs: 179-181 or 182-184, respectively, or sequences with at least about 90%, at least about 91%, at least about 92%, at least about 93%, at least about 94%, at least about 95%, at least about 96%, at least about 97%, at least about 98%, or at least about 99% sequence identity thereto; and (b) CDRH1, CDRH2, and CDRH3 of SEQ ID NOs: 185-187, respectively, or sequences with at least about 90%, at least about 91%, at least about 92%, at least about 93%, at least about 94%, at least about 95%, at least about 96%, at least about 97%, at least about 98%, or at least about 99% sequence identity thereto.

In an embodiment, the antibody or antibody fragment comprises an immunoglobulin light chain variable region comprising the following CDRs: (a) SEQ ID NOs: 1-3, (b) SEQ ID NOs: 7-9, (c) SEQ ID NOs: 13-15, (d) SEQ ID NOs: 19-21, (e) SEQ ID NOs: 25-27, (0 SEQ ID NOs: 31-33, (g) SEQ ID NOs: 37-39, (h) SEQ ID NOs: 43-45, (i) SEQ ID NOs: 49, 44, and 50, (j) SEQ ID NOs: 54-56, (k) SEQ ID NOs: 60-62, or a sequence with at least about 90%, at least about 91%, at least about 92%, at least about 93%, at least about 94%, at least about 95%, at least about 96%, at least about 97%, at least about 98%, or at least about 99% sequence identity thereto.

In an embodiment, the antibody or antibody fragment comprises an immunoglobulin heavy chain variable region comprising the following CDRs: (a) SEQ ID NOs: 4-6, (b) SEQ ID NOs: 10-12, (c) SEQ ID NOs: 16-18, (d) SEQ ID NOs: 22-24, (e) SEQ ID NOs: 28-30, (f) SEQ ID NOs: 34-36, (g) SEQ ID NOs: 40-42, (h) SEQ ID NOs: 46-48, (i) SEQ ID NOs: 51-53, (j) SEQ ID NOs: 57-59, (k) SEQ ID NOs: 63-65, or a sequence with at least about 90%, at least about 91%, at least about 92%, at least about 93%, at least about 94%, at least about 95%, at least about 96%, at least about 97%, at least about 98%, or at least about 99% sequence identity thereto.

Thus, in another embodiment, the antibody or antibody fragment comprises a set of six CDRs, CDRL1, CDRL2, CDRL3, CDRH1, CDRH2, and CDRH3 comprising, respectively: (a) SEQ ID NOs: 1-6, (b) SEQ ID NOs: 7-12, (c) SEQ ID NOs: 13-18, (d) SEQ ID NOs: 19-24, (e) SEQ ID NOs: 25-30,

(f) SEQ ID NOs: 31-36, (g) SEQ ID NOs: 37-42, (h) SEQ ID NOs: 43-48, (i) SEQ ID NOs: 49-53 and 44, (j) SEQ ID NOs: 54-59, (k) SEQ ID NOs: 60-65, or a sequence with at least about 90%, at least about 91%, at least about 92%, at least about 93%, at least about 94%, at least about 95%, at least about 96%, at least about 97%, at least about 98%, or at least about 99% sequence identity thereto.

In an embodiment, the antibody or antibody fragment comprises:

(1) an immunoglobulin light chain variable region comprising a LCDR1 comprising or consisting of SEQ ID NO: 1, a LCDR2 comprising or consisting of SEQ ID NO: 2, and/or a LCDR3 comprising or consisting of SEQ ID NO: 3; and/or an immunoglobulin heavy chain variable region comprising a HCDR1 comprising or consisting of SEQ ID NO: 4, a HCDR2 comprising or consisting of SEQ ID NO: 5, and/or a HCDR3 comprising or consisting of SEQ ID NO: 6;

(2) an immunoglobulin light chain variable region comprising a LCDR1 comprising or consisting of SEQ ID NO: 7, a LCDR2 comprising or consisting of SEQ ID NO: 8, and/or a LCDR3 comprising or consisting of SEQ ID NO: 9; and/or an immunoglobulin heavy chain variable region comprising a HCDR1 comprising or consisting of SEQ ID NO: 10, a HCDR2 comprising or consisting of SEQ ID NO: 11, and/or a HCDR3 comprising or consisting of SEQ ID NO: 12;

(3) an immunoglobulin light chain variable region comprising a LCDR1 comprising or consisting of SEQ ID NO: 13, a LCDR2 comprising or consisting of SEQ ID NO: 14, and/or a LCDR3 comprising or consisting of SEQ ID NO: 15; and/or an immunoglobulin heavy chain variable region comprising a HCDR1 comprising or consisting of SEQ ID NO: 16, a HCDR2 comprising or consisting of SEQ ID NO: 17, and/or a HCDR3 comprising or consisting of SEQ ID NO: 18;

(4) an immunoglobulin light chain variable region comprising a LCDR1 comprising or consisting of SEQ ID NO: 19, a LCDR2 comprising or consisting of SEQ ID NO: 20, and/or a LCDR3 comprising or consisting of SEQ ID NO: 21; and/or an immunoglobulin heavy chain variable region comprising a HCDR1 comprising or consisting of SEQ ID NO: 22, a HCDR2 comprising or consisting of SEQ ID NO: 23, and/or a HCDR3 comprising or consisting of SEQ ID NO: 24;

(5) an immunoglobulin light chain variable region comprising a LCDR1 comprising or consisting of SEQ ID NO: 25, a LCDR2 comprising or consisting of SEQ ID NO: 26, and/or a LCDR3 comprising or consisting of SEQ ID NO: 27; and/or an immunoglobulin heavy chain variable region comprising a HCDR1 comprising or consisting of SEQ ID NO: 28, a HCDR2 comprising or consisting of SEQ ID NO: 29, and/or a HCDR3 comprising or consisting of SEQ ID NO: 30;

(6) an immunoglobulin light chain variable region comprising a LCDR1 comprising or consisting of SEQ ID NO: 31, a LCDR2 comprising or consisting of SEQ ID NO: 32, and/or a LCDR3 comprising or consisting of SEQ ID NO: 33; and/or an immunoglobulin heavy chain variable region comprising a HCDR1 comprising or consisting of SEQ ID NO: 34, a HCDR2 comprising or consisting of SEQ ID NO: 35, and/or a HCDR3 comprising or consisting of SEQ ID NO: 36;

(7) an immunoglobulin light chain variable region comprising a LCDR1 comprising or consisting of SEQ ID NO: 37, a LCDR2 comprising or consisting of SEQ ID NO: 38, and/or a LCDR3 comprising or consisting of SEQ ID NO: 39; and/or an immunoglobulin heavy chain variable region comprising a HCDR1 comprising or consisting of SEQ ID NO: 40, a HCDR2 comprising or consisting of SEQ ID NO: 41, and/or a HCDR3 comprising or consisting of SEQ ID NO: 42;

(8) an immunoglobulin light chain variable region comprising a LCDR1 comprising or consisting of SEQ ID NO: 43, a LCDR2 comprising or consisting of SEQ ID NO: 44, and/or a LCDR3 comprising or consisting of SEQ ID NO: 45; and/or an immunoglobulin heavy chain variable region comprising a HCDR1 comprising or consisting of SEQ ID NO: 46, a HCDR2 comprising or consisting of SEQ ID NO: 47, and/or a HCDR3 comprising or consisting of SEQ ID NO: 48;

(9) an immunoglobulin light chain variable region comprising a LCDR1 comprising or consisting of SEQ ID NO: 49, a LCDR2 comprising or consisting of SEQ ID NO: 44, and/or a LCDR3 comprising or consisting of SEQ ID NO: 50; and/or an immunoglobulin heavy chain variable region comprising a HCDR1 comprising or consisting of SEQ ID NO: 51, a HCDR2 comprising or consisting of SEQ ID NO: 52, and/or a HCDR3 comprising or consisting of SEQ ID NO: 53;

(10) an immunoglobulin light chain variable region comprising a LCDR1 comprising or consisting of SEQ ID NO: 54, a LCDR2 comprising or consisting of SEQ ID NO: 55, and/or a LCDR3 comprising or consisting of SEQ ID NO: 56; and/or an immunoglobulin heavy chain variable region comprising a HCDR1 comprising or consisting of SEQ ID NO: 57, a HCDR2 comprising or consisting of SEQ ID NO: 58, and/or a HCDR3 comprising or consisting of SEQ ID NO: 59;

(11) an immunoglobulin light chain variable region comprising a LCDR1 comprising or consisting of SEQ ID NO: 60, a LCDR2 comprising or consisting of SEQ ID NO: 61, and/or a LCDR3 comprising or consisting of SEQ ID NO: 62; and/or an immunoglobulin heavy chain variable region comprising a HCDR1 comprising or consisting of SEQ ID NO: 63, a HCDR2 comprising or consisting of SEQ ID NO: 64, and/or a HCDR3 comprising or consisting of SEQ ID NO: 65; and/or

(12) an immunoglobulin light chain variable region comprising a LCDR1 comprising or consisting of a sequence of Table 1, a LCDR2 comprising or consisting of a sequence of Table 1, and/or a LCDR3 comprising or consisting of a sequence of Table 1; and/or an immunoglobulin heavy chain variable region comprising a HCDR1 comprising or consisting of a sequence of Table 1, a HCDR2 comprising or consisting of a sequence of Table 1, and/or a HCDR3 comprising or consisting of a sequence of Table 1.

The heavy and light chain variable regions can further comprise any suitable framework regions. In an embodiment, the antibody or antibody fragment comprises:

(1) an immunoglobulin light chain variable region comprising a LFR1 comprising or consisting of SEQ ID NO: 88, a LFR2 comprising or consisting of SEQ ID NO: 89, a LFR3 comprising or consisting of SEQ ID NO: 90, and/or a LFR4 comprising or consisting of SEQ ID NO: 91; and/or an immunoglobulin heavy chain variable region comprising a HFR1 comprising or consisting of SEQ ID NO: 92, a HFR2 comprising or consisting of SEQ ID NO: 93, a HFR3 comprising or consisting of SEQ ID NO: 94, and/or a HFR4 comprising or consisting of SEQ ID NO: 95;

(2) an immunoglobulin light chain variable region comprising a LFR1 comprising or consisting of SEQ ID NO: 96, a LFR2 comprising or consisting of SEQ ID NO: 97, a LFR3 comprising or consisting of SEQ ID NO: 98, and/or a LFR4 comprising or consisting of SEQ ID NO: 99; and/or an immunoglobulin heavy chain variable region comprising a HFR1 comprising or consisting of SEQ ID NO: 100, a HFR2 comprising or consisting of SEQ ID NO: 101, a HFR3 comprising or consisting of SEQ ID NO: 102, and/or a HFR4 comprising or consisting of SEQ ID NO: 103;

(3) an immunoglobulin light chain variable region comprising a LFR1 comprising or consisting of SEQ ID NO: 104, a LFR2 comprising or consisting of SEQ ID NO: 105, a LFR3 comprising or consisting of SEQ ID NO: 106, and/or a LFR4 comprising or consisting of SEQ ID NO: 107; and/or an immunoglobulin heavy chain variable region comprising a HFR1 comprising or consisting of SEQ ID NO: 108, a HFR2 comprising or consisting of SEQ ID NO: 109, a HFR3 comprising or consisting of SEQ ID NO: 110, and/or a HFR4 comprising or consisting of SEQ ID NO: 111;

(4) an immunoglobulin light chain variable region comprising a LFR1 comprising or consisting of SEQ ID NO: 112, a LFR2 comprising or consisting of SEQ ID NO: 113, a LFR3 comprising or consisting of SEQ ID NO: 114, and/or a LFR4 comprising or consisting of SEQ ID NO: 115; and/or an immunoglobulin heavy chain variable region comprising a HFR1 comprising or consisting of SEQ ID NO: 116, a HFR2 comprising or consisting of SEQ ID NO: 117, a HFR3 comprising or consisting of SEQ ID NO: 118, and/or a HFR4 comprising or consisting of SEQ ID NO: 119;

(5) an immunoglobulin light chain variable region comprising a LFR1 comprising or consisting of SEQ ID NO: 120, a LFR2 comprising or consisting of SEQ ID NO: 121, a LFR3 comprising or consisting of SEQ ID NO: 122, and/or a LFR4 comprising or consisting of SEQ ID NO: 123; and/or an immunoglobulin heavy chain variable region comprising a HFR1 comprising or consisting of SEQ ID NO: 124, a HFR2 comprising or consisting of SEQ ID NO: 125, a HFR3 comprising or consisting of SEQ ID NO: 126, and/or a HFR4 comprising or consisting of SEQ ID NO: 127;

(6) an immunoglobulin light chain variable region comprising a LFR1 comprising or consisting of SEQ ID NO: 128, a LFR2 comprising or consisting of SEQ ID NO: 129, a LFR3 comprising or consisting of SEQ ID NO: 130, and/or a LFR4 comprising or consisting of SEQ ID NO: 131; and/or an immunoglobulin heavy chain variable region comprising a HFR1 comprising or consisting of SEQ ID NO: 132, a HFR2 comprising or consisting of SEQ ID NO: 133, a HFR3 comprising or consisting of SEQ ID NO: 134, and/or a HFR4 comprising or consisting of SEQ ID NO: 135;

(7) an immunoglobulin light chain variable region comprising a LFR1 comprising or consisting of SEQ ID NO: 136, a LFR2 comprising or consisting of SEQ ID NO: 137, a LFR3 comprising or consisting of SEQ ID NO: 138, and/or a LFR4 comprising or consisting of SEQ ID NO: 139; and/or an immunoglobulin heavy chain variable region comprising a HFR1 comprising or consisting of SEQ ID NO: 140, a HFR2 comprising or consisting of SEQ ID NO: 141, a HFR3 comprising or consisting of SEQ ID NO: 142, and/or a HFR4 comprising or consisting of SEQ ID NO: 143;

(8) an immunoglobulin light chain variable region comprising a LFR1 comprising or consisting of SEQ ID NO: 144, a LFR2 comprising or consisting of SEQ ID NO: 145, a LFR3 comprising or consisting of SEQ ID NO: 146, and/or a LFR4 comprising or consisting of SEQ ID NO: 147; and/or an immunoglobulin heavy chain variable region comprising a HFR1 comprising or consisting of SEQ ID NO: 148, a HFR2 comprising or consisting of SEQ ID NO: 149, a HFR3 comprising or consisting of SEQ ID NO: 150, and/or a HFR4 comprising or consisting of SEQ ID NO: 151;

(9) an immunoglobulin light chain variable region comprising a LFR1 comprising or consisting of SEQ ID NO: 152, a LFR2 comprising or consisting of SEQ ID NO: 153, a LFR3 comprising or consisting of SEQ ID NO: 154, and/or a LFR4 comprising or consisting of SEQ ID NO: 155; and/or an immunoglobulin heavy chain variable region comprising a HFR1 comprising or consisting of SEQ ID NO: 156, a HFR2 comprising or consisting of SEQ ID NO: 157, a HFR3 comprising or consisting of SEQ ID NO: 158, and/or a HFR4 comprising or consisting of SEQ ID NO: 159;

(10) an immunoglobulin light chain variable region comprising a LFR1 comprising or consisting of SEQ ID NO: 160, a LFR2 comprising or consisting of SEQ ID NO: 161, a LFR3 comprising or consisting of SEQ ID NO: 162, and/or a LFR4 comprising or consisting of SEQ ID NO: 163; and/or an immunoglobulin heavy chain variable region comprising a HFR1 comprising or consisting of SEQ ID NO: 164, a HFR2 comprising or consisting of SEQ ID NO: 165, a HFR3 comprising or consisting of SEQ ID NO: 166, and/or a HFR4 comprising or consisting of SEQ ID NO: 167; and/or

(11) an immunoglobulin light chain variable region comprising a LFR1 comprising or consisting of SEQ ID NO: 168, a LFR2 comprising or consisting of SEQ ID NO: 169, a LFR3 comprising or consisting of SEQ ID NO: 170, and/or a LFR4 comprising or consisting of SEQ ID NO: 171; and/or an immunoglobulin heavy chain variable region comprising a HFR1 comprising or consisting of SEQ ID NO: 172, a HFR2 comprising or consisting of SEQ ID NO: 173, a HFR3 comprising or consisting of SEQ ID NO: 174, and/or a HFR4 comprising or consisting of SEQ ID NO: 175. comprising or consisting of SEQ ID NO: 173, a HFR3 comprising or consisting of SEQ ID NO: 174, and/or a HFR4 comprising or consisting of SEQ ID NO: 175.

In an embodiment, the antibody or antibody fragment comprises an immunoglobulin light chain variable region of any one of SEQ ID NO: 66, 68, 70, 72, 74, 76, 78, 80, 82, 84, and 86, a sequence that is at least about 90%, at least about 91%, at least about 92%, at least about 93%, at least about 94%, at least about 95%, at least about 96%, at least about 97%, at least about 98%, or at least about 99% identical to SEQ ID NO: 66, 68, 70, 72, 74, 76, 78, 80, 82, 84, and 86, or at least the CDRs thereof; and/or an immunoglobulin heavy chain variable region of any one of SEQ ID NO: 67, 69, 71, 73, 75, 77, 79, 81, 83, 85, and 87, or a sequence that is at least about 90%, at least about 91%, at least about 92%, at least about 93%, at least about 94%, at least about 95%, at least about 96%, at least about 97%, at least about 98%, or at least about 99% identical to SEQ ID NO: 67, 69, 71, 73, 75, 77, 79, 81, 83, 85, and 87, or at least the CDRs thereof.

By way of further illustration, the antibody or antibody fragment can comprise:

(1) an immunoglobulin light chain variable region of SEQ ID NO: 66, or at least the CDRs thereof, and/or an immunoglobulin heavy chain variable region of SEQ ID NOs: 67, or at least the CDRs thereof;

(2) an immunoglobulin light chain variable region of SEQ ID NO: 68, or at least the CDRs thereof, and/or an immunoglobulin heavy chain variable region of SEQ ID NOs: 69, or at least the CDRs thereof;

(3) an immunoglobulin light chain variable region of SEQ ID NO: 70, or at least the CDRs thereof, and/or an immunoglobulin heavy chain variable region of SEQ ID NOs: 71, or at least the CDRs thereof;

(4) an immunoglobulin light chain variable region of SEQ ID NO: 72, or at least the CDRs thereof, and/or an immunoglobulin heavy chain variable region of SEQ ID NOs: 73, or at least the CDRs thereof;

(5) an immunoglobulin light chain variable region of SEQ ID NO: 74, or at least the CDRs thereof, and/or an immunoglobulin heavy chain variable region of SEQ ID NOs: 75, or at least the CDRs thereof;

(6) an immunoglobulin light chain variable region of SEQ ID NO: 76, or at least the CDRs thereof, and/or an immunoglobulin heavy chain variable region of SEQ ID NOs: 77, or at least the CDRs thereof;

(7) an immunoglobulin light chain variable region of SEQ ID NO: 78, or at least the CDRs thereof, and/or an immunoglobulin heavy chain variable region of SEQ ID NOs: 79, or at least the CDRs thereof;

(8) an immunoglobulin light chain variable region of SEQ ID NO: 80, or at least the CDRs thereof, and/or an immunoglobulin heavy chain variable region of SEQ ID NOs: 81, or at least the CDRs thereof;

(9) an immunoglobulin light chain variable region of SEQ ID NO: 82, or at least the CDRs thereof, and/or an immunoglobulin heavy chain variable region of SEQ ID NOs: 83, or at least the CDRs thereof;

(10) an immunoglobulin light chain variable region of SEQ ID NO: 84, or at least the CDRs thereof, and/or an immunoglobulin heavy chain variable region of SEQ ID NOs: 85, or at least the CDRs thereof;

(11) an immunoglobulin light chain variable region of SEQ ID NO: 86, or at least the CDRs thereof, and/or an immunoglobulin heavy chain variable region of SEQ ID NOs: 87, or at least the CDRs thereof, or

(12) an immunoglobulin light chain variable region of Table 1, or at least the CDRs thereof, and/or an immunoglobulin heavy chain variable region of Table 1, or at least the CDRs thereof.

In connection with any of the embodiments provided herein, the CDRs of a given Ig sequence, such as the heavy and light chain sequences mentioned herein, can be determined by any of several conventional numbering schemes, such as Kabat, Chothia, Martin (Enhanced Chothia), IGMT, or AHo (see, e.g., Kabat, et al., Sequences of Proteins of Immunological Interest, U.S. Department of Health and Human Services, NIH (1991); Chothia, et al., Canonical Structures for the Hypervariable Regions of Immunoglobulins, J. Mol. Biol., 196:901-917 (1987); Al-Lazikani et al., Standard Conformations for the Canonical Structures of Immunoglobulins, J. Mol. Biol., 273:927-948 (1997); Abhinandan et al., Analysis and Improvements to Kabat and Structurally Correct Numbering of Antibody Variable Domains, Mol. Immunol., 45: 3832-3839 (2008); Lefranc et al., The IMGT unique numbering for immunoglobulins, T cell Receptors and Ig-like domains, The Immunologist, 7: 132-136 (1999); Lefranc et al., IMGT unique numbering for immunoglobulin and T cell receptor variable domains and I superfamily V-like domains, Dev. Comp. Immunol., 27: 55-77 (2003); and Honegger et al., Yet another numbering scheme for immunoglobulin variable domains: an automatic modeling and analysis tool, J. Mol. Biol. 309: 657-670 (2001). In a particular embodiment, the CDRs can be any of those specific CDR sequences provided herein.

In an embodiment, the antibody or antibody fragment comprises the set of CDRs and/or heavy chain variable region and/or light chain variable region of Table 1.

TABLE 1

| | | | | | | | Light | Heavy |
|---|---|---|---|---|---|---|---|---|
| | | | | | | | Chain | Chain |
| | | | | | | | Variable | Variable |
| Clone | CDRL1 | CDRL2 | CDRL3 | CDRH1 | CDRH2 | CDRH3 | Region | Region |
| 3N02 | SEQ ID | SEQ ID | SEQ ID | SEQ ID | SEQ ID | SEQ ID | SEQ ID | SEQ ID |
| | NO: 1 | NO: 2 | NO: 3 | NO: 4 | NO: 5 | NO: 6 | NO: 66 | NO: 67 |
| 4S04 | SEQ ID | SEQ ID | SEQ ID | SEQ ID | SEQ ID | SEQ ID | SEQ ID | SEQ ID |
| | NO: 19 | NO: 20 | NO: 21 | NO: 22 | NO: 23 | NO: 24 | NO: 72 | NO: 73 |
| 4S12 | SEQ ID | SEQ ID | SEQ ID | SEQ ID | SEQ ID | SEQ ID | SEQ ID | SEQ ID |
| | NO: 54 | NO: 55 | NO: 56 | NO: 57 | NO: 58 | NO: 59 | NO: 84 | NO: 85 |
| 3NS2 | SEQ ID | SEQ ID | SEQ ID | SEQ ID | SEQ ID | SEQ ID | SEQ ID | SEQ ID |
| | NO: 13 | NO: 14 | NO: 15 | NO: 16 | NO: 17 | NO: 18 | NO: 70 | NO: 71 |
| 3NS1 | SEQ ID | SEQ ID | SEQ ID | SEQ ID | SEQ ID | SEQ ID | SEQ ID | SEQ ID |
| | NO: 37 | NO: 38 | NO: 39 | NO: 40 | NO: 41 | NO: 42 | NO: 78 | NO: 79 |
| 3NS3 | SEQ ID | SEQ ID | SEQ ID | SEQ ID | SEQ ID | SEQ ID | SEQ ID | SEQ ID |
| | NO: 43 | NO: 44 | NO: 45 | NO: 46 | NO: 47 | NO: 48 | NO: 80 | NO: 81 |
| 4S10 | SEQ ID | SEQ ID | SEQ ID | SEQ ID | SEQ ID | SEQ ID | SEQ ID | SEQ ID |
| | NO: 49 | NO: 44 | NO: 50 | NO: 51 | NO: 52 | NO: 53 | NO: 82 | NO: 83 |
| 5S8 | SEQ ID | SEQ ID | SEQ ID | SEQ ID | SEQ ID | SEQ ID | SEQ ID | SEQ ID |
| | NO: 25 | NO: 26 | NO: 27 | NO: 28 | NO: 29 | NO: 30 | NO: 74 | NO: 75 |
| 3N07 | SEQ ID | SEQ ID | SEQ ID | SEQ ID | SEQ ID | SEQ ID | SEQ ID | SEQ ID |
| | NO: 7 | NO: 8 | NO: 9 | NO: 10 | NO: 11 | NO: 12 | NO: 68 | NO: 69 |
| d301 | SEQ ID | SEQ ID | SEQ ID | SEQ ID | SEQ ID | SEQ ID | SEQ ID | SEQ ID |
| | NO: 31 | NO: 32 | NO: 33 | NO: 34 | NO: 35 | NO: 36 | NO: 76 | NO: 77 |
| MN66 | SEQ ID | SEQ ID | SEQ ID | SEQ ID | SEQ ID | SEQ ID | SEQ ID | SEQ ID |
| | NO: 60 | NO: 61 | NO: 62 | NO: 63 | NO: 64 | NO: 65 | NO: 86 | NO: 87 |

Nucleic acid or amino acid sequence “identity,” as referenced herein, can be determined by comparing a nucleic acid or amino acid sequence of interest to a reference nucleic acid or amino acid sequence. The percent identity is the number of nucleotides or amino acid residues that are the same (i.e., that are identical) as between the aligned sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, BLASTp, BLASTn, and the like) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)). Percent (%) identity of sequences can be also calculated, for example, as 100×[(identical positions)/min(TG_{A}, TG_{B})], where TG_{A} and TG_{B} are the sum of the number of residues and internal gap positions in peptide sequences A and B in the alignment that minimizes TG_{A} and TG_{B}. See, e.g., Russell et al., J. Mol Biol., 244: 332-350 (1994).

The amino acids of the sequences provided can be substituted with any other amino acid. Amino acids include naturally-occurring α-amino acids and their stereoisomers, as well as non-naturally occurring amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypiation) or deglycosylated.

Naturally-occurring amino acids are those encoded by the genetic code, as well as those amino acids that are later modified, e.g., hydroxyproline, γ-carboxyglutamate, and O-phosphoserine. Naturally-occurring a-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring a-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Non-naturally occurring amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission.

The amino acid substitution can be conservative, semi-conservative, or non-conservative with respect to the basic properties of the original amino acid residue. A “conservative” substitution refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (D or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained.

“Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In addition, one or more amino acids can be inserted into the aforementioned immunoglobulin heavy chain polypeptides. Any number of any suitable amino acids can be inserted into the amino acid sequence of the immunoglobulin heavy chain polypeptide. In this respect, at least one amino acid (e.g., 2 or more, 5 or more, or 10 or more amino acids), but not more than 20 amino acids (e.g., 18 or less, 15 or less, or 12 or less amino acids), can be inserted into the amino acid sequence of the immunoglobulin heavy chain polypeptide. Preferably, 1-10 amino acids (e.g., 1, 2, 3, 4, 5, 6, 7, 8, 9, or 10 amino acids) are inserted into the amino acid sequence of the immunoglobulin heavy chain polypeptide. In this respect, the amino acid(s) can be inserted into any one of the aforementioned immunoglobulin heavy chain polypeptides in any suitable location. In some embodiments, the amino acid(s) are inserted into a CDR (e.g., CDR1, CDR2, or CDR3) of the immunoglobulin heavy chain polypeptide; in other embodiments, the amino acids are inserted into a framework region.

The antibody can be a complete (full) antibody, or an antigen binding antibody fragment. The antibody may be of any immunoglobulin type (e.g., IgG, IgE, IgM, IgD, or IgA), or class (e.g., IgG1, IgG2, IgG3, or IgG4). The antigen binding fragment can be any part of an antibody that has at least one antigen binding site, including, but not limited to, IgGΔCH₂, Fab, F(ab′)2, Fv, dsFv, scFv, scFv2CH3, scFv4, scFv3, scFv2, scFv-Fc, diabodies, triabodies, bis-scFvs, (scFv)2, fragments expressed by a Fab expression library, domain antibodies, VHH domains, V-NAR domains, VH domains, VL domains, and the like. Antibody fragments of the invention, however, are not limited to these exemplary types of antibody fragments. Furthermore, the antibody or antibody fragment can be engineered to have various configurations known in the art. For example, the antibody or antibody fragment can be linked to a synthetic molecule with the following domains: a spacer or hinge region (e.g., a CD28, CD28, or IgG hinge), a transmembrane region (e.g., a transmembrane canonical domain), and/or an intracellular T-cell receptor (TCR) signaling domain, thereby forming a T-body or chimeric antigen receptor (CAR). Intracellular TCR signaling domains that can be included in a T-body (or CAR) include, but are not limited to, CD3, FcR-γ, and Syk-PTK signaling domains as well as the CD28, 4-1BB, and CD134 co-signaling domains. Methods for constructing T-cells expressing a T-body (or CAR) are known in the art. See, e.g., Marcu-Malina et al., Expert Opinion on Biological Therapy, 9: 539-564 (2009).

The antibody or antibody fragment includes antibodies that have been mutated or otherwise modified to modulate function. For instance, the antibody or antibody fragment can comprise a mutation of the Fc-region of a human IgG1 heavy chain to enhance effector function, as described in WO 2013/004842. Or, the antibody can be glycoengineered, for instance, to enhance monocyte/macrophage-mediated phagocytosis and cytotoxicity (see, e.g., Herter et al., J. Immunol., 192(5): 2252-60 (2014). The antibody or antibody fragments described herein can be modified in any of various other ways known in the art without departing from the scope of the invention.

A domain antibody comprises a functional binding unit of an antibody, and can correspond to the variable regions of either the heavy (VH) or light (VL) chains of antibodies. A domain antibody can have a molecular weight of approximately 13 kDa, or approximately one-tenth of a full antibody. Domain antibodies may be derived from full antibodies such as those described herein.

The antigen binding fragments in some embodiments are monomeric or polymeric, bispecific or trispecific, bivalent or trivalent. Antibody fragments that contain the antigen binding, or idiotype, of the antibody molecule may be generated by techniques known in the art. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab′ fragments which may be generated by treating the antibody molecule with papain and a reducing agent.

A single-chain variable region fragment (scFv) antibody fragment, which consists of a truncated Fab fragment comprising the variable (V) domain of an antibody heavy chain linked to a V domain of a light antibody chain via a synthetic peptide, can be generated using routine recombinant DNA technology techniques (see, e.g., Janeway et al. (eds.), Immunobiology, 5th Ed., Garland Publishing, New York, N.Y. (2001)). Similarly, disulfide -stabilized variable region fragments (dsFv) can be prepared by recombinant DNA technology (see, e.g., Reiter et al., Protein Engineering, 7, 697-704 (1994)).

Recombinant antibody fragments, e.g., scFvs, can also be engineered to assemble into stable multimeric oligomers of high binding avidity and specificity to different target antigens. Such diabodies (dimers), triabodies (trimers) or tetrabodies (tetramers) are well known in the art, see e.g., Kortt et al., Biomol. Eng., 18: 95-108, (2001) and Todorovska et al., J. Immunol. Methods, 248: 47-66 (2001).

Bispecific antibodies (bscAb) are molecules comprising two single-chain Fv fragments joined via a glycine-serine linker using recombinant methods. The V light-chain (VL) and V heavy-chain (VH) domains of two antibodies of interest in exemplary embodiments are isolated using standard PCR methods. The VL and VH cDNAs are then joined to form a single-chain fragment in a two-step fusion PCR. Bispecific fusion proteins are prepared in a similar manner. Bispecific single-chain antibodies and bispecific fusion proteins are antibody substances included within the scope of the present invention. Exemplary bispecific antibodies are taught in U.S. Patent Application Publication No. 2005-0282233A1 and International Patent Application Publication No. WO 2005/087812, both applications of which are incorporated herein by reference in their entirety. The multispecific antibody can be configured as a BiTE or DART. BiTEs consist of a single polypeptide displaying two antigen-binding specificities through cognate heavy and light chain variable domains. BiTEs have one N-terminus and one C-terminus. In DARTs, cognate heavy and light chain variable domains are on two separate polypeptides that associate and are stabilized by a C-terminal disulfide bridge. Thus, DARTs have 2 N-termini and 2 C-termini.

The anti-C3d antibody can be made by any suitable technique. The antibody is an engineered antibody produced by synthetic, recombinant, or other manufacturing techniques. Suitable methods of making engineered antibodies are known in the art. For instance, a polyclonal antibody can be prepared by immunizing an animal with an immunogen (e.g., C3d) and collecting antisera from that immunized animal. A wide range of animal species can be used for the production of antisera. In some aspects, an animal used for production of antisera is a non-human animal including rabbits, mice, rats, hamsters, goat, sheep, pigs or horses. Polyclonal antisera may be obtained, after allowing time for antibody generation, simply by bleeding the animal and preparing serum samples from the whole blood. The polyclonal antibodies, thus, obtained can then be screened for specific desired antibodies (e.g., antibodies of the invention).

Monoclonal antibodies may be prepared using any technique which provides for the production of antibody molecules by continuous cell lines in culture. Several hybridoma methods are known in the art (e.g., Koehler and Milstein, Nature, 256: 495-497 (1975); Kosbor et al., Immunol Today, 4: 72 (1983); Cote et al., Proc. Natl. Acad. Sci., 80: 2026-2030, 1983); Cole et al., Monoclonal Antibodies and Cancer Therapy, Alan R Liss Inc, New York N.Y., pp 77-96, (1985); Harlow and Lane (eds.), Antibodies: A Laboratory Manual, CSH Press (1988), and CA. Janeway et al. (eds.), Immunobiology, 5th Ed., Garland Publishing, New York, N.Y. (2001); Haskard and Archer, J. Immunol. Methods, 74(2): 361-67 (1984); Roder et al., Methods Enzymol., 121: 140-67 (1986); Huse et al., Science, 246: 1275-81 (1989)). Other known antibody production techniques can also be used, such as by producing human antibodies in non-human animals (e.g., U.S. Pat. Nos. 5,545,806, 5,569,825, and 5,714,352, and U.S. Patent Application Publication No. 2002/0197266), screening methods (e.g., Orlandi et al., Proc. Natl. Acad. Sci., 86: 3833-3837 (1989), and Winter et al., Nature, 349: 293-299 (1991); phage display methods (e.g., Sambrook et al. (eds.), Molecular Cloning, A Laboratory Manual, 3 Edition, Cold Spring Harbor Laboratory Press, New York (2001); use of transgenic mice (e.g., U.S. Pat. Nos. 5,545,806 and 5,569,825).

Methods for generating engineered and humanized antibodies are well known in the art (e.g., Janeway et al. (eds.), Immunobiology, 5th Ed., Garland Publishing, New York, N.Y. (2001); U.S. Pat. Nos. 5,225,539, 5,585,089, 5,693,761, and 5,693,762; European Patent No. 0239400 B1, and United Kingdom Patent No. 2188638; Jones et al., Nature, 321: 522-525 (1986); Riechmann et al., Nature, 332: 323-327 (1988) and Verhoeyen et al., Science 239: 1534-1536 (1988); U.S. Pat. No. 5,639,641; Pedersen et al., J. Mol. Biol., 235: 959-973 (1994); and Owens and Young, J. Immunol. Meth., 168: 149-165 (1994).

Techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci., 81: 6851-6855 (1984); Neuberger et al., Nature, 312: 604-608 (1984); Takeda et al., Nature, 314: 452-454 (1985)). Also, techniques described for the production of single chain antibodies can be employed (U.S. Pat. No. 4,946,778). If a preferred embodiment, the antibodies of the invention are chimeric rabbit/human antibodies.

Chemically constructed bispecific antibodies may be prepared by chemically cross-linking heterologous Fab or F(ab′)2 fragments by means of chemicals such as heterobifunctional reagent succinimidyl-3-(2-pyridyldithiol)-propionate (SPDP, Pierce Chemicals, Rockford, Ill.). The Fab and F(ab′)2 fragments can be obtained from intact antibody by digesting it with papain or pepsin, respectively (Karpovsky et al., J. Exp. Med. 160: 1686-701 (1984); Titus et al., J. Immunol., 138: 4018-22 (1987)).

Methods of testing antibodies for the ability to bind to C3d, regardless of how the antibodies are produced, are known in the art and include any antibody-antigen binding assay, such as, for example, radioimmunoassay (RIA), enzyme-linked immunosorbent assay (ELISA), Western blot, immunoprecipitation, and competitive inhibition assays (see, e.g., Janeway et al. (eds.), Immunobiology, 5th Ed., Garland Publishing, New York, N.Y. (2001); and U.S. Patent Application Publication No. 2002/0197266 A1).

The antibody can be isolated. The term “isolated” as used herein encompasses compounds or compositions that have been removed from a biological environment (e.g., a cell, tissue, culture medium, body fluid, etc.), or otherwise increased in purity to any degree (e.g., isolated from a synthesis medium). Isolated compounds and compositions, thus, can be synthetic or naturally produced.

Also provided is a nucleic acid encoding the anti-C3d antibody as described herein, which can be used to produce the antibody by expressing the nucleic acid in a cell. The nucleic acid can comprise any suitable nucleotide sequence that encodes the antibody or portion thereof (e.g., CDRs, framework regions, and other parts of the antibody or antibody fragment). A nucleic acid comprising the desired nucleotide sequence can be constructed based on chemical synthesis and/or enzymatic ligation reactions using procedures known in the art (e.g., The nucleic acids in some aspects are constructed based on chemical synthesis and/or enzymatic ligation reactions using procedures known in the art. See, for example, et al. (eds.), Molecular Cloning, A Laboratory Manual, 3 Edition, Cold Spring Harbor Laboratory Press, New York (2001).

Also provided is a recombinant expression vector comprising the nucleotide sequence encoding the antibody or antibody fragment. The recombinant expression vector of the invention can be any suitable recombinant expression vector, and can be used to transform or transfect any suitable host. Suitable vectors include those designed for propagation and expansion or for expression or both, such as plasmids and viruses. Examples of vectors include the pUC series (Fermentas Life Sciences), the pBluescript series (Stratagene, La Jolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λ [symbol] TIO, λ [symbol] TI 1, AZapII (Stratagene), EMBL4, and λNMI 149, also can be used. Examples of plant expression vectors include pBIO1, pBI101.2, pBI101.3, pBI121 and pBIN19 (Clontech). Examples of animal expression vectors include pEUK-Cl, pMAM and pMAMneo (Clontech). The recombinant expression vector can be a viral vector, e.g., a retroviral vector.

Constructs of expression vectors, which are circular or linear, can be prepared to contain a replication system functional in a prokaryotic or eukaryotic host cell. Replication systems can be derived, e.g., from CoIEl, 2μ, plasmid, λ, SV40, bovine papilloma virus, and the like.

The recombinant expression vector can comprise regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector may include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the presently disclosed expression vectors include, for instance, neomycin/G418 resistance genes, hygromycin resistance genes, histidinol resistance genes, tetracycline resistance genes, and ampicillin resistance genes.

The recombinant expression vector can comprise a native or non-native promoter operably linked to the nucleotide sequence encoding the polypeptide (including functional portions and functional variants thereof), or to the nucleotide sequence which is complementary to or which hybridizes to the nucleotide sequence encoding the polypeptide. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, and a promoter found in the long-terminal repeat of the murine stem cell virus.

The recombinant expression vectors of the invention can be prepared using standard recombinant DNA techniques described in, for example, Sambrook et al., supra.

The nucleic acid or vector can be in a host cell. The host cell can be any type of cell. The host cell in some aspects is a eukaryotic cell, e.g., plant, animal, fungi, or algae, especially a human cell, or can be a prokaryotic cell, e.g., bacteria or protozoa. The host cell in some aspects is a cultured cell or a primary cell, i.e., isolated directly from an organism, e.g., a human. The host cell in some aspects is an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like.

Thus, the invention further provides eukaryotic or non-eukaryotic cells that have been recombinantly engineered to produce an antibody or antibody fragment of the invention. The cells can be targeted immune cells that are engineered to recombinantly express the anti-C3d antibody or antibody fragment as a cell surface reactive antibody or antibody fragment, such as a T-body or chimeric antigen receptor (CAR). For example, cell can be a T-cell engineered to express an antibody or antibody fragment of the invention (e.g., an scFv, scFv-Fc, or (scFv)2) linked to a spacer or hinge region (e.g., a CD28, CD28, or IgG hinge), a transmembrane region (e.g., a transmembrane canonical domain), and an intracellular T-cell receptor (TCR) signaling domain, thereby forming a T-body or CAR. Intracellular TCR signaling domains that can be included in a T-body (or CAR) include, but are not limited to, CD3ζ, FcR-γ, and Syk-PTK signaling domains as well as the CD28, 4-1BB, and CD134 co-signaling domains. Methods for constructing T-cells expressing a T-body (or CAR) are known in the art. See, e.g., Marcu-Malina et al., Expert Opinion on Biological Therapy, 9: 539-564 (2009).

The anti-C3d antibody or fragment thereof may be conjugated or fused to another molecule, or to a support, optionally by way of a linker molecule. Any of a variety of molecules can be conjugated or fused to the anti-C3d antibody for various purposes, including diagnostic, marking or tracing, therapeutic, or recovery/purification purposes. Examples of such other molecules include, without limitation, detectable labels, affinity tags, and therapeutic agents, including cytotoxic, cytostatic, or antiangiogenic agents and radioisotopes. Therapeutic agents can be, for example, a plant, fungal, or bacterial molecules (e.g., a protein toxin), small molecule chemotherapeutics, or biological therapeutics. Examples of therapeutic molecules include, for instance, a maytansinoid (e.g., maytansinol or DM1 maytansinoid), a taxane, a calicheamicin, an antimetabolite (e.g., an antifolate such as methotrexate, a fluoropyrimidine such as 5-fluorouracil, cytosine arabinoside, or an analogue of purine or adenosine); an intercalating agent (for example, an anthracycline such as doxorubicin, daunomycin, epirubicin, idarubicin, mitomycin-C, dactinomycin, or mithramycin); a platinum derivative (e.g., cisplatin or carboplatin); an alkylating agent (e.g., nitrogen mustard, melphalan, chlorambucil, busulphan, cyclophosphamide, ifosfamide nitrosoureas, or thiotepa); an antimitotic agent (e.g., a vinca alkaloid like vincristine or taxoid such as paclitaxel or docetaxel); a topoisomerase inhibitor (for example, etoposide, and teniposide, amsacrine, or topotecan); a cell cycle inhibitor (for example, a flavopyridol); a microbtubule agent (e.g., an epothilone, discodermolide analog, or eleutherobin analog); a proteosome inhibitor or a topoisomerase inhibitor such as bortezomib, amsacrine, etoposide, etoposide phosphate, teniposide, or doxorubicin; a radioisotope including yttrium (⁹⁰Y), lutetium (¹⁷⁷Lu), actinium (²²⁵Ac), praseodymium, astatine (²¹¹At), rhenium (¹⁸⁶Re) bismuth (²¹²Bi or ²¹³Bi), and rhodium (¹⁸⁸Rh); an antiangiogenic agent such as linomide, bevacizumab, angiostatin, and razoxane; an antibody or antibody fragment other than an anti-C3d antibody or antibody fragment, such as rituximab or bevacizumab. Labels can be useful in diagnostic applications and can include, for example, radiolabels contrast agents. A contrast agent can be a radioisotope label such as iodine (¹³¹I or ¹²⁵I), indium (¹¹¹In), technetium (⁹⁹Tc), phosphorus (³²P), carbon (¹⁴C), tritium (³H), other radioisotope (e.g., a radioactive ion), or a therapeutic radioisotope listed above. Additionally, contrast agents can include radiopaque materials, magnetic resonance imaging (MRI) agents, ultrasound imaging agents, and any other contrast agents suitable for detection by a device that images an animal body; as well as a fluorescent label, a biologically active enzyme label, a luminescent label, or a chromophore label.

Methods of conjugating or fusing such other molecules to an antibody without interfering with the binding of the antibody to its target antigen are known in the art. Recombinant engineering and incorporated selenocysteine (e.g., as described in International Patent Application Publication WO 2008/122039) can be used to conjugate a synthetic molecule. Other methods of conjugation can include covalent coupling to native or engineered lysine side-chain amines or cysteine side-chain thiols. See, e.g., Wu et al., Nat. Biotechnol., 23: 1137-1146 (2005).

The anti-C3d antibody or antibody fragment can be part of a composition, particularly a pharmaceutical composition, comprising the anti-C3d antibody or fragment and a carrier. Any carrier suitable for proteins, particularly antibodies, can be used. A pharmaceutically acceptable carrier is preferred. The term “pharmaceutically acceptable carrier” as used herein means one or more compatible solid or liquid fillers, diluents, other excipients, or encapsulating substances which are suitable for administration into a human or veterinary patient. The carrier can be co-mingled with the one or more active components without substantially impairing the desired pharmaceutical efficacy. Pharmaceutically acceptable materials generally are capable of administration to a patient without the production of significant undesirable physiological effects. The pharmaceutical composition can contain suitable buffering agents, preservatives, and other components typically used in pharmaceutical formulations, particularly therapeutic antibody formulations. The pharmaceutical composition can be presented in a unit dosage form suitable for the desired route of administration (e.g., oral, parenteral, etc).

The anti-C3d antibody can be used for any purpose, such as for labeling opsonized cells, or targeting opsonized cells for delivery of a therapeutic agent. Thus, the invention provides, in one aspect, a method of labeling a cell comprising C3d complement protein on the surface thereof (e.g., an opsonized cell) by contacting the cell with an anti-C3d antibody as described herein that contains a detectable label. According to another aspect, the invention provides a method of delivering a therapeutic agent to a cell comprising C3d complement protein on the surface thereof (e.g., an opsonized cell) by contacting the cell with an anti-C3d antibody as described herein attached to a therapeutic agent. All aspects of the anti-C3d antibody attached to a detectable label or therapeutic agent are as previously described.

The anti-C3d antibody is believed to be particularly useful for eliminating cells, especially cancer cells, by binding to C3d surface proteins on such cells and causing their destruction by cell lysis or phagocytosis. Thus, the invention further provides a method of killing cancer cells comprising C3d complement protein on the surface thereof (e.g., a C3d opsonized, viable cancer cell) by contacting the cell with the anti-C3d antibody described herein, wherein the immune system of the subject is recruited to kill the cancer cell. Alternatively, the method can comprise administering anti-C3d antibody conjugated to a cytotoxic agent to the subject, wherein the anti-C3d antibody targets cancer cells with C3d on the surface and the cytotoxic agent kills the cells. In this respect, killing cancer cells is not limited to direct killing of cancer cells, but includes any method or mechanism by which a living, viable cancer cell may be eliminated from a host as a result of contacting the cancer cell with an antibody of the invention. The invention further provides a method of reducing or eliminating metastasis.

The cell may be any type of cell having a C3d surface protein. Typically, the cell will be a cancer cell or other pathogenic cell having a C3d surface protein (e.g., an opsonized cancer cell or other pathogenic cell). The cancer cell may acquire C3d surface proteins through binding of a mAb to molecules on the surface of the cancer cell. The cancer cell can be a cell of any type of cancer that has a C3d protein on the cell surface. Non-limiting examples of specific types of cancers include cancer of the head and neck, eye, skin, mouth, throat, esophagus, chest, bone, lung, colon, sigmoid, rectum, stomach, prostate, breast, ovaries, uterus (e.g., endometrium), kidney, liver, pancreas, brain, intestine, heart or adrenals. More particularly, cancers include solid tumor, sarcoma, carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor, acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute undifferentiated leukemia, chronic myelocytic leukemia, chronic lymphocytic leukemia, hairy cell leukemia, or multiple myeloma. See, e.g., Harrison's Principles of Internal Medicine, Eugene Braunwald et al., eds., pp. 491 762 (15th ed. 2001).

The methods of the invention are believed to be especially useful to target a cancer cell that has lost a therapeutic target antigen through trogocytosis. In a specific embodiment, the cell is a chronic lymphocytic leukemia cell, and the method may be performed on a patient with chronic lymphocytic leukemia. In another embodiment, the cancer cell is a B-cell expressing CD20, and the method may be performed on a patient with CD20+ B-cell malignancy. In yet another embodiment, the cell is from a cancer treated with a mAb and the method may be performed on a patient in need of treatment for such a cancer.

Any of the foregoing methods may further comprise inducing the formation of C3d on the surface of the cell. This may be accomplished by contacting the cell with an agent that activates the complement system, such as by opsonizing the cell by contact with an antibody or antigen-binding fragment thereof that binds to a surface protein on the cell other than C3d. The antibody that binds to a cell-surface protein other than C3d can be, for example, a therapeutic antibody or antibody fragment. Examples include an anti-CD20 antibody or antibody fragment (e.g., rituximab, ofatumumab, ocrelizumab, veltuzumab, obinutuzumab (GA-101), PRO131921, or ocaratuzumab (AME-133)), an anti-CD33 antibody or antibody fragment (e.g., gemtuzumab, or lintuzumab), an anti-CD38 antibody (e.g., daratumumab), an anti-CD52 antibody (e.g., Alemtuzumab), an anti-ERBB2 antibody (e.g., trastuzumab), and an anti-EGFR antibody (e.g., cetuximab, or panitumumab).

The anti-C3d antibody or antibody fragment and the antibody or antibody fragment that binds to a surface protein on the cell other than C3d can be separate molecules or they can be part of the same multi-specific antibody. Thus, for instance, a multi-specific antibody having immunospecificity for C3d and a different cell-surface protein (e.g., CD20, CD33, CD38, etc.) can be used to both induce the complement cascade resulting in C3d formation on the cell surface and to bind C3d once formed. If separate antibodies or antibody fragments are used to induce C3d formation on the cell surface and bind to C3d once formed, they can be administered to the subject simultaneously or sequentially in any order, though typically the antibody that binds to a cell-surface protein other than C3d will be administered before or approximately at the same time as the anti-C3d antibody.

In accordance with any of the foregoing methods, the cell may be in vitro or in vivo. For instance, the cell may be in a patient, who may be afflicted with a disease. When the cell is in the patient, the cell may be contacted by the anti-C3d antibody or other agent by administering the antibody or other agent to the patient. If the patient is afflicted with a disease, the administration of the anti-C3d antibody and/or other agents can, according to certain embodiments, treat the disease by reducing one or more symptoms or characteristics of the disease. The patient can be a patient treated with a therapy (e.g., monoclonal antibody therapy, particularly anti-CD20 monoclonal antibody therapy, such as with rituximab or ofatumumab) resulting in C3d deposition on the cancer cells. In one embodiment trogocytosis may lead to loss of the CD20 antigen and anti-C3d mAb can be used to target these cells. In a second embodiment anti CD20 therapy leads to C3d deposition but incomplete killing of the cancer cell, that can then be eliminated by anti-C3d mAb alone, or by the combination of the anti CD20 mAb with the anti-C3d mAb. In one embodiment anti-CD20 mAb is combined with anti-C3d mAb for the treatment of CD20 positive B-cell malignancies.

Thus, for instance, the method may comprise administering to a patient, simultaneously or sequentially, an anti-C3d antibody as described herein and an agent that induces formation of C3d on the surface of a cell (e.g., a monoclonal antibody, such as an anti-CD20 antibody like rituximab, ofatumumab, ocrelizumab, veltuzumab, obinutuzumab (GA-101), PRO131921, ocaratuzumab (AME-133) or any antibody or antibody fragment to a cell surface antigen described herein, or other opsonizing agent). The cell is particularly a pathogenic cell such as a cancer cell. By way of further example, the cell can be a chronic lymphocytic leukemia cell, and the patient can have chronic lymphocytic leukemia.

The anti-C3d antibody and agent that induces formation of C3d on the surface of a cell can be administered simultaneously as separate compositions or as a single composition, or the two agents can be administered sequentially in any order. When administered sequentially, the timeframe of administration is not particularly limited, but the anti-C3d antibody and agent that induces C3d formation on the cell will typically be administered within several minutes (e.g., within 10, 20, 30, 40, or 50 minutes) or within several hours (e.g., within 1, 2, 4, 8, 12, or 24 hours), or within several days (e.g., 1, 2, 5, 7), or within several weeks (e.g., 1, 2, 3, 4) of one another.

Also provided herein is a short (about 50 amino acids or less) polypeptide comprising an anti-C3d epitope of SEQ ID NO: 192, 193, or a combination thereof. The polypeptide can have fewer than 50 amino acids, such as about 45 amino acids or fewer, about 40 amino acids or fewer, about 35 amino acids or fewer, about 30 amino acids or fewer, about 25 amino acids or fewer, or about 20 amino acids or fewer. In some embodiments, the polypeptide can consist essentially of, or consist of, SEQ ID NO: 192 or 193, or combination thereof. If the polypeptide comprises amino acid residues flanking the sequence of SEQ ID NO: 192 or 193, or combination thereof, the flanking residues can have any suitable sequence provided they do not interfere with the binding of the epitope sequence to an antibody targeting the epitope. The flanking sequences can be, for instance, the sequences that flank SEQ ID NOs: 192 or 193 in the native C3d protein, such that the polypeptide is a fragment of C3d comprising SEQ ID NO: 192, 193, or a combination of such fragments joined together.

In yet another embodiment, the polypeptide can comprise, consist essentially of, or consist of a fragment of SEQ ID NO: 192, 193, or a combination of such fragments, large enough to facilitate binding of an antibody (e.g., an anti-C3d antibody as described herein). Thus, the fragment will typically comprise at least about 5 contiguous amino acids, such as at least about 10 contiguous amino acids, of SEQ ID NO: 192 or 193.

The polypeptide can be bound to a support, directly or via a linker molecule. The support can be any type of support (e.g., solid supports, such as a bead or plate), particular a support useful in biopanning techniques, such as panning a phage display library.

The polypeptide can be used for any suitable purpose. For instance, the polypeptide can be used to screen for, select, or produce anti-C3d antibodies. Thus, provided herein is a method of screening, selecting, or producing anti-C3d antibodies by contacting one or more antibodies or antibody fragments (e.g., a library, such as a phage display library) with the polypeptide and selecting an antibody or antibody fragment that binds to the polypeptide. The method can comprise repeatedly performing the contacting and selection steps (e.g., panning) using the polypeptide and selecting those antibodies or antibody fragments that exhibit the greatest affinity for the polypeptide. Specific techniques for panning antibody libraries using polypeptides are known in the art. For instance, the polypeptide can be used in conjunction with panning phage display libraries.

The polypeptides also can be used to elicit an immunogenic response in a mammal. Thus, provided herein is a method of eliciting an immunogenic response in a mammal by administering the polypeptide to a mammal. The immunogenic response can be for any purpose, such as for therapy or for the production and subsequent harvesting of antibodies.

Also provided is a nucleic acid encoding the polypeptide, optionally in a vector. The vector can be any of those described herein with respect to the nucleic acid encoding the antibody of the invention.

The following examples further illustrate the invention but, of course, should not be construed as in any way limiting its scope.

### EXAMPLE 1

This example demonstrates the production and characterization of antibodies of the invention.

Cell Lines

HEK293F cells (Thermo Fisher Scientific, Waltham, Mass.) were maintained in FreeStyle Medium without fetal bovine serum (FBS) for suspension culture, and with 100 U/mL penicillin, and 100 mg/mL streptomycin (Thermo Fisher Scientific). JeKo-1 cells from American Type Culture Collection (ATCC) were cultured in RPMI-1640 (Thermo Fisher Scientific) supplemented with 10% (v/v) heat inactivated FBS (Thermo Fisher Scientific), 100 U/mL penicillin, and 100 mg/mL streptomycin (Thermo Fisher Scientific).

C3d Proteins

Human complement protein C3d (hC3d) (Complement Technology, Inc., Tyler, Tex.) was used for immunization. DNA sequence of hC3d (aa1002-1303 of hC3) with a mutation at position 1010 from cysteine to alanine to avoid thioester bond formation with glutamine at position 1013 was custom synthesized as gBlock and cloned into pCEP4-hFc via HindIII/XhoI (see Yang, et al., PLoS One, 6: e21018 (2011)). Similarly, mouse C3d (mC3d) with the same mutation was cloned to pCEP4-hFc as well. The resulting pCEP4-hFc-hC3d and pCEP4-hFc-mC3d plasmids were confirmed by DNA sequencing and transiently transfected into HEK293F cells using polyethylenimine (PEI). Transfected cells were cultured in FREESTYLE™ protein-free medium and the hFc-hC3d and hFc-mC3d proteins were purified from supernatants by Protein A affinity chromatography (see Yang, et al., PLoS One, 6: e21018 (2011)). The quality and quantity of purified proteins were analyzed by SDS-PAGE and Am absorbance, respectively.

Deposition of C3b/iC3b/C3d on Zymosan Particles and JeKo-1 Cells

To deposit C3 fragments on zymosan, one hundred million of preactivated zymosan particles (Complement Technology, Inc.) were washed and then incubated with 50% pooled normal human complement serum (PNHCS) or normal BALB/c mouse or cynomolgus complement serum in 1 mL Dulbecco's phosphate-buffered saline (DPBS) at 37° C. for 1 h. For JeKo-1 cells, 10 μg/mL rituximab (RTX) was first used to opsonize cells followed by incubation with 50% CS-depleted PNHCS. To allow C3b/iC3b deposited on JeKo-1 cells to decay to C3d, the incubation time of cells with serum was prolonged to 24 h, or 0.5 U/mL CR1 (rhCD35, R&D Systems, Minneapolis, Minn.) was included during the incubation. After washing three times (by spinning at 3,000 g for 3 min for sedimentation of zymosan particles), twenty million of the opsonized zymosan particles were used for panning and 1 million of the particles or cells were used for ELISA or flow cytometry.

Generation and Selection of Immune Chimeric Rabbit/Human Fab Libraries by Phage Display

Two b9 allotype rabbits were immunized with 100 μg hC3d and Freund's complete adjuvant, followed by three boosts with 50 μg hC3d and Freund's incomplete adjuvant every three weeks. The antibody response to the immunogen was monitored during the process by ELISA. Spleen and bone marrow from the two b9 rabbits were collected five days after the last boost and separately processed for total RNA preparation and RT-PCR amplification of rbV_{κ}, rbV_{λ}, and rbV_{H} encoding cDNA using established protocols and primers (see Peng, et al., J. Mol. Biol., 429: 2954-2973 (2017)). A degenerate reverse primer rbIgG-C_{H}1-R (5′gaagactganggagccttaggttg3′, n=t or c, SEQ ID NO: 194)) binding to the 5′ end of the rabbit IgG constant domain C_{H}1 was used to enrich all rbV_{H} encoding sequences in the secondary antibody repertoire. Rabbit (rb) V_{κ}/human (hu) C_{κ}/rbV_{H} and rbV_{λ}/huC_{λ}/rbV_{H} segments, respectively, were assembled and cloned into phage display vector pC3C (see Peng, et al., J. Mol. Biol., 429: 2954-2973 (2017)). Transformation of E. coli strain ER2738 (Lucigen Corporation, Middleton, Wis.) by electroporation yielded approximately 8.6×10⁸ and 2.6×10⁹ independent transformants for phagemid library κ and λ, respectively. Using VCSM13 helper phage (Agilent Technologies, Santa Clara, Calif.), the phagemid libraries were converted to phage libraries and stored at −80 ° C. Phage library κ and λ, were re-amplified using ER2738 and mixed equally before four rounds of panning against native C3b/iC3b/C3d deposited on zymosan. Starting from the third round, panning was performed with or without 50% PNHCS when phage was incubated with zymosan particles. Following selection, supernatants of IPTG-induced bacterial clones were analyzed by zymosan ELISA or flow cytometry. Repeated clones were identified by DNA fingerprinting with AluI, and the V_{L} and V_{H} sequences of unique clones were determined by DNA sequencing.

Zymosan ELISA

One million zymosan particles deposited with C3b/iC3b/C3d were added into one well of a V-bottom 96-well plate. Then 100 ng/100 μL of Fab was applied in each well at 37° C. for one hour. After washing three times by PBST (0.05% (v/v) Tween 20 in PBS), 100 μL of a 1:1000 dilution of a mouse anti-His tag mAb conjugated to horseradish peroxidase (HRP) (R&D Systems) in 1% (w/v) BSA/PBS was used to detect the Fab. To test the binding of antibodies to native C3b/iC3b/C3d deposited on zymosan in the presence of C3 in serum, serial dilutions of human serum were premixed with: (1) zymosan particles prior to addition of 1 μg/mL of each Fab, or (2) each Fab at 15 μg/mL before adding to zymosan particles deposited with human C3 fragments. To determine whether the chimeric rabbit/human Fabs have overlap binding sites over chimeric mouse/human anti-human C3d mAb C8xi, serial dilutions of C8xi IgG1 were premixed with each anitbody at 1 μg/mL before adding to zymosan particles deposited with human C3 fragments. Colorimetric detection was performed using 2,2′-azino-bis (3-ethylbenzthiazoline)-6-sulfonic acid (F. Hoffmann-La Roche AG, Basel, Switzerland) as substrate according to the manufacturer's directions. The absorbance was measured at 405 nm using a SPECTRAMAX™ M5 microplate reader (Molecular Devices, LLC, San Jose, Calif.) and SOFTMAX™ Pro software (Molecular Devices).

Zymosan Flow Cytometry

One million zymosan particles deposited with C3b/iC3b/C3d were added into one well of a V-bottom 96-well plate. Then 500 ng/100 μL of antibody was applied in each well at 37° C. for 1 h. After washing three times by phosphate buffered saline and TWEEN™ 20 (PBST), 100 μL of a 1:1000 dilution of a mouse anti-His tag mAb conjugated to ALEXA FLUOR™ 488 (Qiagen, Hilden, Germany) in 1% (w/v) bovine serum albumin (BSA)/DPBS was used to detect the antibody.

Surface Plasmon Resonance

Surface plasmon resonance (SPR) for the measurement of kinetic and thermodynamic parameters of the binding of purified antibodies to hC3d or mC3d antigens was performed on a Biacore X100 instrument using Biacore reagents and software (GE Healthcare, Chicago, Ill.). A mouse anti-human IgG C_{H}2 mAb was immobilized on a CM5 sensor chip using reagents and instructions supplied with the Human Antibody Capture Kit (GE Healthcare). hFc-hC3d and hFc-mC3d fusion proteins were captured at a density not exceeding 400 RU. Each sensor chip included an empty flow cell for instantaneous background depletion. All binding assays used 1×HBS-EP+ running buffer (10 mM HEPES, 150 mM NaCl, 3 mM EDTA (pH 7.4), and 0.05% (v/v) surfactant P20) and a flow rate of 30 μL/min. All of the Fabs were injected at five different concentrations (dilution factor was 2), and the lowest concentration was tested in duplicates. The sensor chips were regenerated with 3 M MgCl₂ from the Human Antibody Capture Kit without any loss of binding capacity. Calculation of association (kₒₙ) and dissociation (k_{off}) rate constants was based on a 1:1 Langmuir binding model. The equilibrium dissociation constant (K_{D}) was calculated from k_{off}/kₒₙ.

Table 2 provides a summary of the properties of the antibodies of the invention.

TABLE 2

| | | | | | Zymosan | | | | |
|---|---|---|---|---|---|---|---|---|---|
| | | | | | deposited | | | | |
| | | | | | C3b/iC3b/(C3d) | | | | |
| | | | Kd (nM) | Kd (nM) | | Cyno | | Jeko1 cell line | Human |
| Clones | Ag | Isotype | hFc-hC3d | hFc-mC3d | Human | monkey | mouse | deposited C3d* | serum C3 |
| C8xi | hC3d | K | +++ | − | +++ | +++ | − | NA | (+) |
| (prior art) | | | | | | | | | |
| 3N02 | Zymosan- | K | 13.3 | 97.6 | +++ | +++ | +++ | NA | +++ |
| | C3b/iC3b/C3d | | | | | | | | |
| 4S04 | Zymosan- | K | 4.11 | − | +++ | +++ | − | NA | ++ |
| | C3b/iC3b/C3d | | | | | | | | |
| 4S12 | Zymosan- | λ | − | − | ++ | − | − | NA | +++ |
| | C3b/iC3b/C3d | | | | | | | | |
| 3NS2 | Zymosan- | λ | − | − | +++ | ++ | − | NA | ++ |
| | C3b/iC3b/C3d | | | | | | | | |
| 3NS1 | Zymosan- | λ | − | − | +++ | +++ | − | + | − |
| | C3b/iC3b/C3d | | | | | | | | |
| 3NS3 | Zymosan- | λ | − | − | ++ | + | − | NA | + |
| | C3b/iC3b/C3d | | | | | | | | |
| 4S10 | Zymosan- | λ | − | − | +++ | +++ | + | NA | − |
| | C3b/iC3b/C3d | | | | | | | | |
| 5S8 | Zymosan- | K | 3.35 | 9.04 | +++ | +++ | +++ | +++ | (+) |
| | C3b/iC3b/C3d | | | | | | | | |
| 3N07 | Zymosan- | K | 3.10 | 132 | +++ | +++ | ++ | +++ | + |
| | C3b/iC3b/C3d | | | | | | | | |
| d301 | Zymosan- | K | 0.99 | 22.9 | +++ | +++ | +++ | +++ | + |
| | C3b/iC3b/C3d | | | | | | | | |
| MN66 | hFc-hC3d | λ | 0.31 | − | +++ | +++ | ++ | NA | NA |

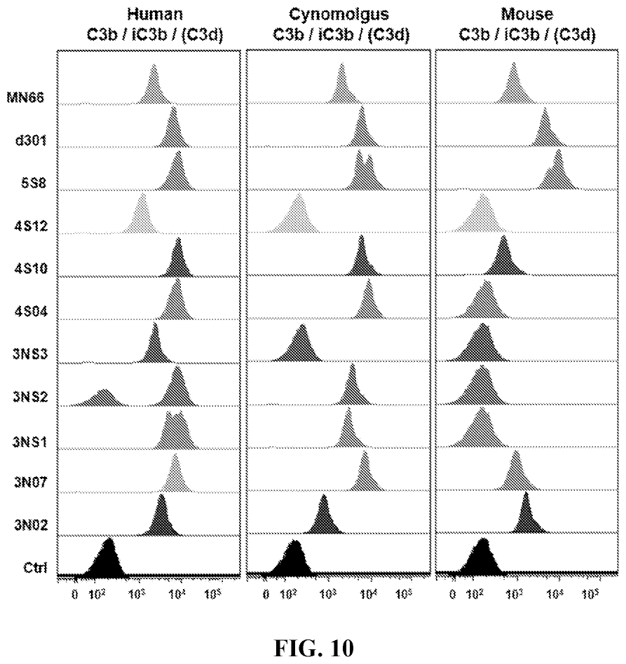

Table 2 notes: “NA” indicates not analyzed; “*” Jeko1 cells deposited with C3b/iC3b/C3d were treated by CR1 or opsonized for 24 h to allow full C3b/iC3b decay to C3d (primary data depicted in FIG. 10); result shown indicates binding specifically to C3d only; relative level of signal is shown by the number of plus or minus signs (e.g., +++=high affinity).

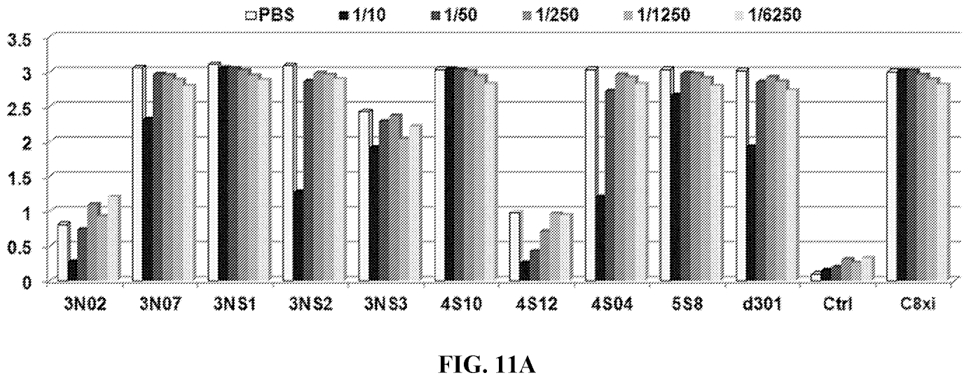

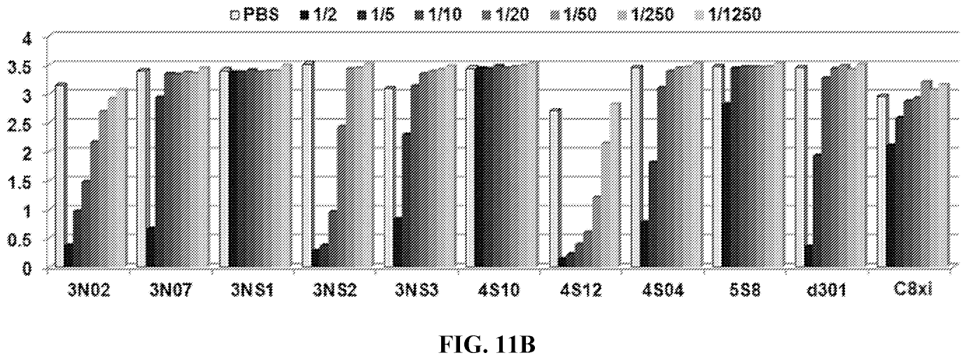

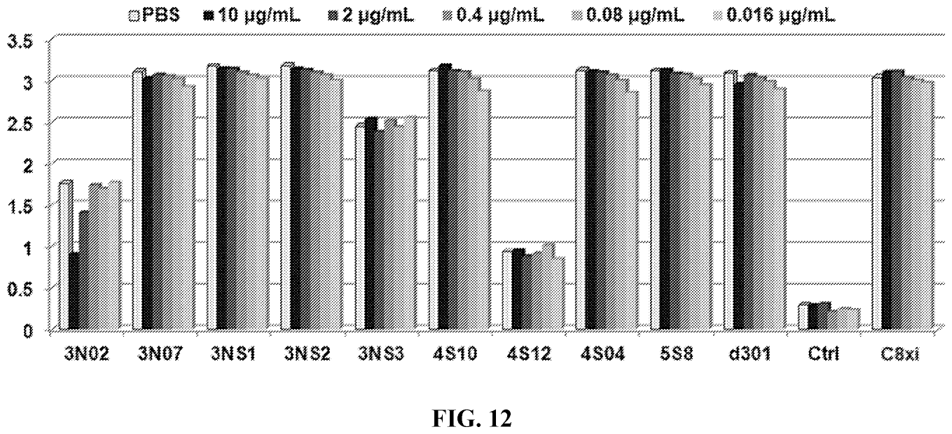

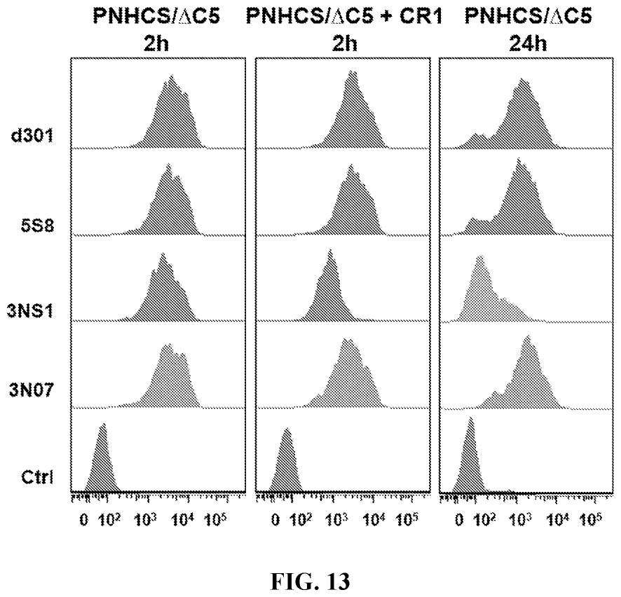

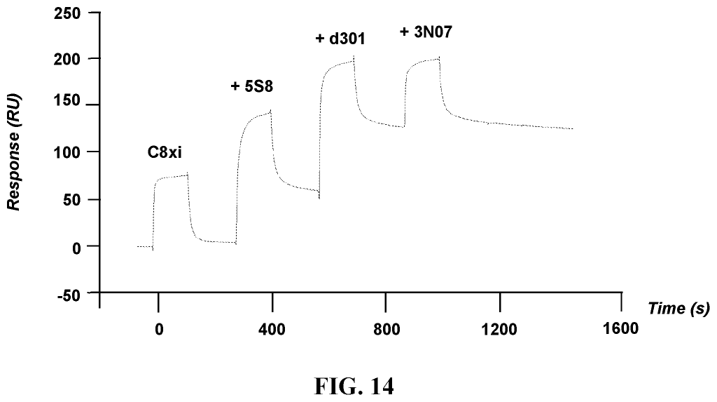

FIGS. 11A-11B are graphs showing the level of competition binding of antibodies of the invention and C8xi to human C3 fragments deposited on zymosan and C3 in human serum. FIG. 12 is a graph showing the level of competition binding of chimeric rabbit/human Fabs with chimeric mouse/human IgG C8xi to native human C3 fragments. FIG. 13 is a graph showing the binding activity of antibodies of the invention to different human C3 fragments. FIG. 14 is a graph showing epitope binning of selected anti C3d Fabs by SPR. The increase found for the binding of Fab 5S8 exceeded the values found for C8xi (prior art) alone, indicating that Fab 5S8 and C8xi can bind simultaneously to hC3d. Similarly binding of Fab d301 exceeded the values found for Fab 5S8 alone indicating that these two antibodies can bind simultaneously to hC3d. By contrast, data indicate that the epitope of Fab 3N07 overlaps with the epitope(s) of Fab d301.

### EXAMPLE 2

This example demonstrates that antibodies of the invention bind to non-human C3d epitopes.

The cross reactivity of 5 μg/mL of antibodies of the invention with 6xHis tag to human, or cynomolgus, or mouse C3b/iC3b/C3d deposited on zymosan particles after incubation with 50% corresponding serum at 37° C. for 45 min was determined by flow cytometry with a secondary mouse anti-HIS mAb conjugated to ALEXA FLUOR™ 488. In vitro binding studies of the antibodies 5S8 and d301 demonstrated that these antibodies have high affinity for macaque and mouse C3d (see Table 3 below and FIG. 10). In Table 3, the relative level of affinity is shown by the number of plus or minus signs (e.g., +++=high affinity). m/h and r/h denote rabbit and mouse, respectively, variable domains fused human constant domains; k, kappa light chain; λ, lambda light chain.

TABLE 3

| | Isoelectric | | Human | Macaque | Mouse |
|---|---|---|---|---|---|
| Clone | point | Epitope | C3d | C3d | C3d |
| C8xi (prior | 8.5 | Hu: | +++ | +++ | - |
| art) | | KDAPDHQELNL | | | |
| m/h-IgG1k | | DVSLQL (SEQ ID | | | |
| | | NO: 188) | | | |
| 5S8 | 7.9 | Not identified, not | +++ | +++ | +++ |
| r/h-IgG1k | | overlapping with | 3.4 nM | | 9.0 nM |
| | | epitope of C8xi | | | |
| d301 | 8.1 | Not overlapping | +++ | +++ | +++ |
| r/h-IgG1k | | with epitope of | 0.99 nM | | 23 nM |
| | | C8xi or 5S8. | | | |
| | | Competes with | | | |
| | | 3N07 | | | |
| 3N02 | | Partially competes | ++ | + | + |
| r/h-IgG1k | | with C8xi | 13 nM | | 98 nM |
| 3NS1 | | Hu: | +++ | ++ | |
| r/h-IgG1λ | | LQEAKDICEEQVN | | | |
| | | (SEQ ID NO: 192) | | | |
| 3N07 r/h- | | Hu: | +++ | +++ | + |
| IgG1k | | QYQKDAP (SEQ | 3.1 nM | | 132 nM |
| | | ID NO: 193) | | | |

This example demonstrates that the binding of the antibodies of the present invention to immobilized C3d is not reduced in the presence of NHS meaning that the antibodies are not competed away by native full-length C3 present in human plasma.

Antibodies 3N02, 3NS1, 5S8, d301, and C8xi were tested at various concentrations of NHS, 1/2, 1/5, 1/10, 1/20, and 1/50 dilutions, and PBS was used as the control. 10 μg/ml of the listed antibody was used.

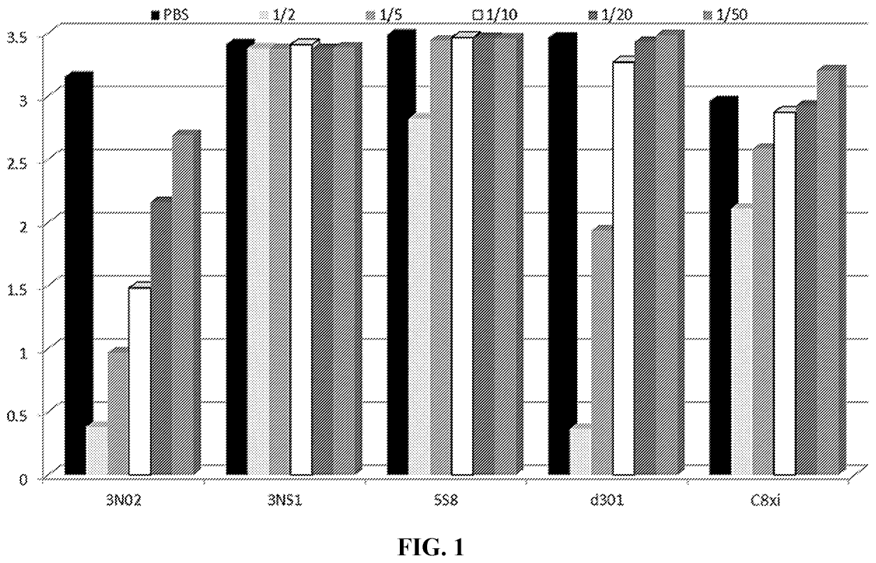

As seen in FIG. 1, 3NS1 and 5S8 were not or only minimally, respectively, reduced in C3d binding in the presence of NHS as the source of full-length C3 protein.

### EXAMPLE 3

This example demonstrates that antibodies of the invention can induce phagocytosis (ADCP) of primary CLL cells.

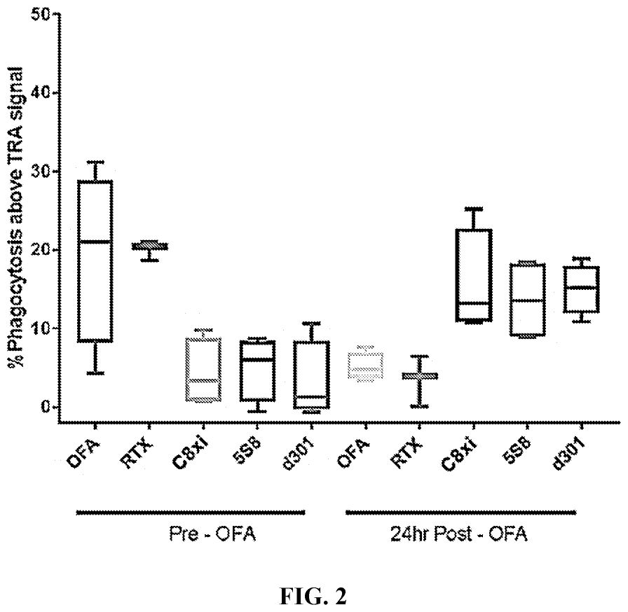

PBMCs were obtained from patients with chronic lymphocytic leukemia before initiation of treatment (Pre-OFA) and in these cells anti-CD20 antibodies (OFA, ofatumumab and RTX, rituximab) effectively induced phagocytosis while anti-C3d antibodies, as expected, had no or minimal effects. In contrast, in PBMCs obtained from patients on day 2, 24 hours after the first infusion of ofatumumab (Post-OFA), CD20 antibodies lost their activity (consistent with documented loss of CD20 by trogocytosis), while all 3 anti-C3d antibodies tested could mediate phagocytosis (see FIG. 2).

### EXAMPLE 4

This example demonstrates that antibodies of the invention are an effective treatment for cancer when combined with an anti-CD20 antibody.

Cell Lines

HBL-2 cells were a gift of Dr. Louis Staudt (NCI), JeKo-1, SUDHL-4, and SUDHL-6 cells were purchased from American Type Culture Collection (ATCC). Cell lines were cultured in RPMI-1640 (Thermo Fisher Scientific) supplemented with 10% (v/v) heat inactivated FBS (Thermo Fisher Scientific), 100 U/mL penicillin, and 100 mg/mL streptomycin (Thermo Fisher Scientific).

Xenograft Lymphoma Model

All the mouse experiments were conducted in compliance with an institutionally approved animal protocol. Cell lines chosen to model Mantle Cell Lymphoma (HBL-2) or Diffuse Large B-cell Lymphoma (SUDHL-4, SUDHL-6) were grown in vitro, washed three times in PBS, passed through 70 u filter and resuspended in injection grade PBS for s.c. injection into Balb/c-SCID mice (The Jackson Laboratory stock #1803). mAbs were injected i.p. and started on day 7 (HBL-2 model), or once palpable tumors were detected in most mice (SUDHL-4 and SUDHL-6 model). Tumor growth was recorded by twice weekly caliper measurements. The mice were euthanized when the tumor volume reached the predefined threshold.

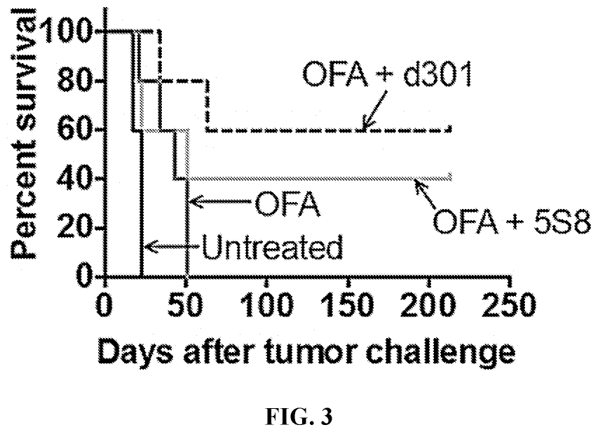

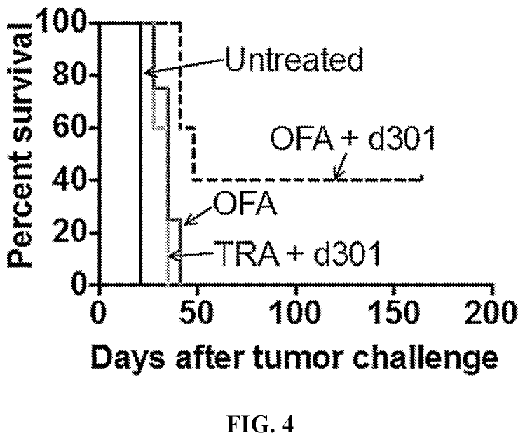

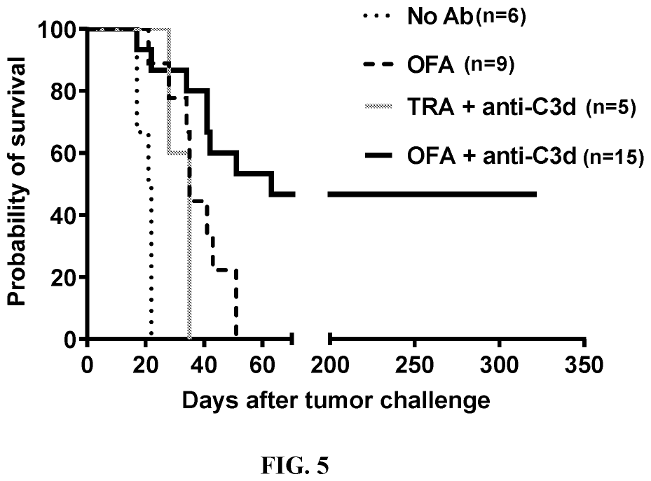

5S8 and d301 were investigated in the HBL-2 lymphoma model in vivo. The anti-CD20 antibody ofatumumab alone extended survival of the mice but all mice succumb to disease. When the anti-CD20 mAb was combined with an anti-C3d mAb survival was significantly improved over ofatumumab single agent therapy (P=0.01) and almost half of the mice survived long-term. Non-targeting control antibody (TRA, trastuzumab) in combination with anti-C3d mAb d301 had minimal anti-tumor effects. Data is summarized in FIGS. 3-5.

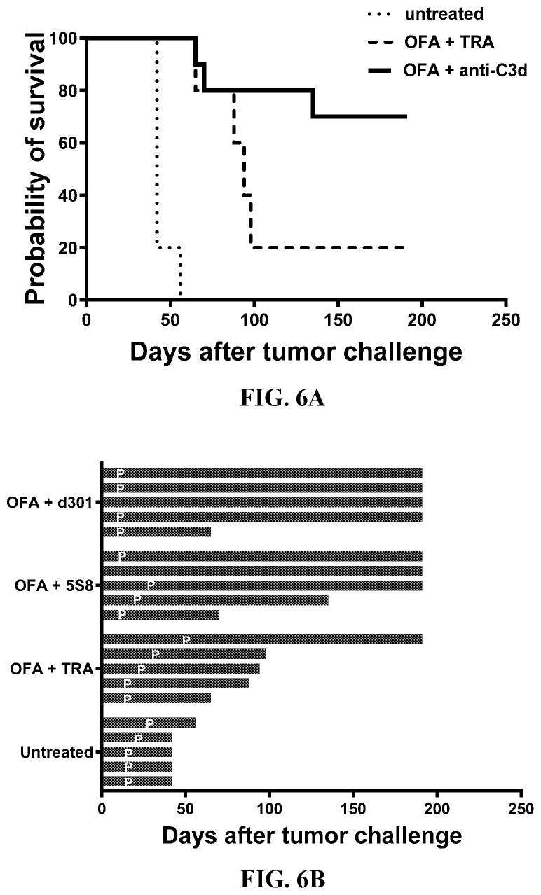

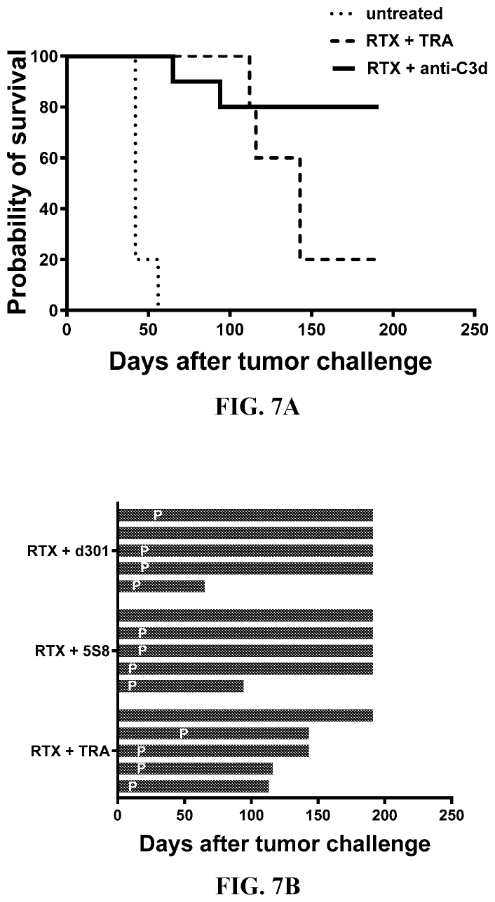

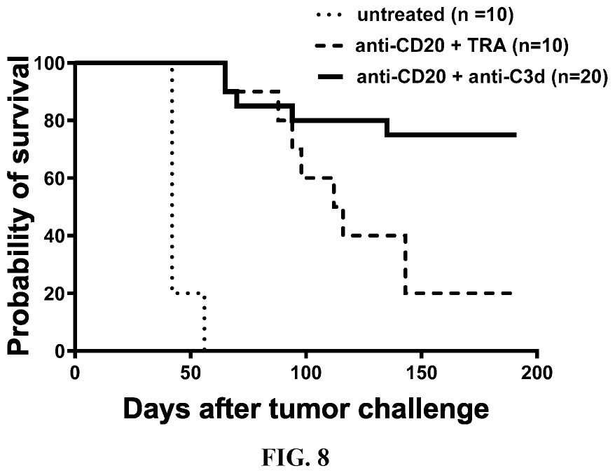

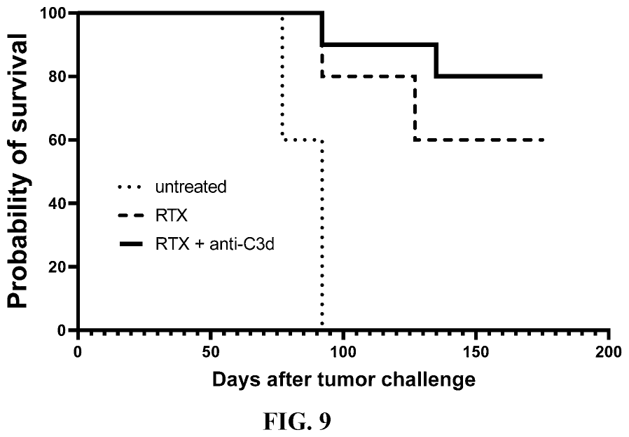

5S8 and d301 were further investigated in the SUDHL-6 model (aggressive lymphoma of the Diffuse Large B-cell Lymphoma type) in vivo. An anti-CD20 antibody (ofatumumab or rituximab) alone extended survival of the mice but most mice still succumbed to disease. When the anti-CD20 mAb was combined with an anti-C3d mAb survival was significantly improved over treatment with anti-CD20 mAb alone and 75% of mice survived long-term (P=0.008). Data is summarized in FIGS. 6A-8.

5S8 and d301 were further investigated in the SUDHL-4 model (aggressive lymphoma of the Diffuse Large B-cell Lymphoma type) in vivo. Rituximab alone achieved long-term disease free-survival in 60% of the mice. When rituximab was combined with an anti-C3d mAb (5S8 or d301) survival improved to 80% but this difference was not statistically significant. Long-term survivors in both treatment arms were sacrificed for necropsy studies that revealed no evidence of renal or retinal toxicity of adding the anti-C3d antibody to rituximab (data summarized in Table 4).

Table 4 provides a summary of analysis of mice surviving long-term after therapy with either rituximab or rituximab with an anti-C3d mAb.

| Mouse I.D. | Treatment | Body Wt. (g) | Creatinine (0.7-1.3) |
|---|---|---|---|
| Y614 | None | 32.0 | 0.26 |
| Y615 | None | 28.5 | 0.26 |
| Y616 | None | 30.3 | 0.32 |
| 2022 | RTX | 31.8 | 0.3 |
| 2023 | RTX | 33.4 | 0.3 |
| 2024 | RTX | 33.5 | 0.3 |
| 2027 | RTX + 5S8 | 30.2 | 0.4 |
| 2028 | RTX + 5S8 | 30.8 | 0.3 |
| 2030 | RTX + 5S8 | 28.9 | 0.3 |
| 2031 | RTX + 5S8 | 28.1 | 0.2 |
| 2032 | RTX + d301 | 32.2 | 0.3 |
| 2034 | RTX + d301 | 27.1 | 0.4 |
| 2035 | RTX + d301 | 23.4 | 0.5 |
| 2036 | RTX + d301 | 29.6 | 0.3 |

Age matched untreated control mice were used for reference values. Hematology profile and a full chemistry panel was done on the blood samples collected from all mice. No abnormality was noted among the groups. Focused histopathologic evaluation revealed no evidence of renal or retinal pathology.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An anti-C3d antibody or antibody fragment comprising:
(a) CDRL1, CDRL2, and CDRL3 comprising SEQ ID NOs: 179-181 or 182-184, respectively, or sequences with at least about 97% sequence identity thereto; and
(b) CDRH1, CDRH2, and CDRH3 comprising SEQ ID NOs: 185-187, respectively, or sequences with at least about 97% sequence identity thereto.

2. The anti-C3d antibody or antibody fragment of claim 1, comprising one of the following sets of complementary determining regions:
(a) SEQ ID NOs: 1-6;
(b) SEQ ID NOs: 7-12;
(c) SEQ ID NOs: 13-18;
(d) SEQ ID NOs: 19-24;
(e) SEQ ID NOs: 25-30;
(f) SEQ ID NOs: 31-36;
(g) SEQ ID NOs: 37-42;
(h) SEQ ID NOs: 43-48;
(i) SEQ ID NOs: 49-53 and 44;
(j) SEQ ID NOs: 54-59;
(k) SEQ ID NOs: 60-65; or
a sequence with at least about 97% sequence identity thereto.

3. The anti-C3d antibody or antibody fragment of claim 1, wherein the antibody or antibody fragment comprises one of the following sets of light chain CDRs:
(a) SEQ ID NOs: 1-3;
(b) SEQ ID NOs: 7-9;
(c) SEQ ID NOs: 13-15;
(d) SEQ ID NOs: 19-21;
(e) SEQ ID NOs: 25-27;
(f) SEQ ID NOs: 31-33;
(g) SEQ ID NOs: 37-39;
(h) SEQ ID NOs: 43-45;
(i) SEQ ID NOs: 49, 44, and 50;
(j) SEQ ID NOs: 54-56;
(k) SEQ ID NOs: 60-62; or
a sequence with at least about 97% sequence identity thereto; or
wherein the anti-C3d antibody or antibody fragment comprises one of the following sets of heavy chain CDRs:
(a) SEQ ID NOs:4-6;
(b) SEQ ID NOs: 10-12;
(c) SEQ ID NOs: 16-18;
(d) SEQ ID NOs: 22-24;
(e) SEQ ID NOs: 28-30;
(f) SEQ ID NOs: 34-36;
(g) SEQ ID NOs: 40-42;
(h) SEQ ID NOs: 46-48;
(i) SEQ ID NOs: 51-53;
(j) SEQ ID NOs: 57-59;
(k) SEQ ID NOs: 63-65; or
a sequence with at least about 97% sequence identity thereto; or
wherein the anti-C3d antibody or antibody fragment comprises at least one chain of the following sets of heavy and light chain variable region polypeptides:
(a) SEQ ID NOs: 66-67;
(b) SEQ ID NOs: 68-69;
(c) SEQ ID NOs: 70-71;
(d) SEQ ID NOs: 72-73;
(e) SEQ ID NOs: 74-75;
(f) SEQ ID NOs: 76-77;
(g) SEQ ID NOs: 78-79;
(h) SEQ ID NOs: 80-81;
(i) SEQ ID NOs: 82-83;
(j) SEQ ID NOs: 84-85;
(k) SEQ ID NOs: 86-87;
or at least the CDRs thereof,
or a sequence with at least about 95% sequence identity thereto.

4-6. (canceled)

7. The antibody or antibody fragment of claim 1, wherein the antibody or antibody fragment is selected from the group consisting of IgA1, IgA2, IgD, IgE, IgG1, IgG2, IgG3, IgG4, IgM, scFv, IgGΔCH2, F(ab′)2, scFv2CH3, F(ab), scFv4, scFv3, scFv2, dsFv, Fv, scFv-Fc, (scFv)2, a diabody, a T-body, a multispecific antibody, and a multivalent antibody.

8. The antibody or antibody fragment of claim 1, wherein the antibody or antibody fragment is conjugated to another molecule.

9. The antibody of claim 8, wherein the antibody or antibody fragment is conjugated to a transmembrane region and an intracellular T-cell receptor (TCR) signaling domain to provide a T-body; or
wherein the anti-C3d antibody or antibody fragment is conjugated to a label; or
wherein the anti-C3d antibody or antibody fragment is conjugated to a cytotoxic agent or a therapeutic radioisotope.

10-11. (canceled)

12. A method of killing a cancer cell having C3d complement protein on the surface thereof, or otherwise treating cancer characterized by surface C3d complement protein, in a subject, the method comprising administering to the subject an anti-C3d antibody or antibody fragment of claim 1.

13. The method of claim 12, further comprising inducing the formation of C3d complement protein on the surface of the cancer cell by contacting the cell with an antibody or antibody fragment to a cell-surface protein other than C3d.

14. The method of claim 13, wherein the anti-C3d antibody is a multi-specific antibody that is immunospecific for C3d and a cell-surface protein other than C3d, and contacting the cell with an antibody or antibody fragment to a cell-surface protein other than C3d is accomplished by administering the multi-specific antibody.

15. The method of claim 13, wherein the method comprises administering to the subject an antibody or antibody fragment that specifically binds to a cell-surface protein other than C3d simultaneously or sequentially in any order with the administration of the anti-C3d antibody or antibody fragment.

16. The method of claim 12, wherein the antibody or antibody fragment to a cell surface protein other than C3d is an anti-CD20 antibody or antibody fragment; or
wherein the antibody or antibody fragment to a cell surface protein other than C3d is rituximab, ofatumumab, ocrelizumab, veltuzumab, obinutuzumab, ublituximab, PRO131921, or ocaratuzumab; or
wherein the antibody or antibody fragment to a cell surface protein other than C3d is an anti-CD33 antibody or antibody fragment; or
wherein the antibody or antibody fragment that specifically binds to a cell surface protein other than C3d is an anti-CD38 antibody or antibody fragment; or
wherein the antibody or antibody fragment to a cell surface protein other than C3d is an anti-CD3 antibody or antibody fragment.

17. (canceled)

18. The method of claim 12, wherein the cancer cell is a B-cell expressing CD20; or
wherein the cancer cell is a myeloid cell; or
wherein the cancer cell is a plasma cell; or
wherein the cancer cell is from gut, colon, lung, breast, head, neck, pancreas, ovary, endometrium, or brain tissue; or
wherein the cancer cell is a T-cell expressing CD3.

19-22. (canceled)

23. The method of claim 12, wherein the method comprises contacting the cancer cell with an anti-EGFR or anti-ERBB2 antibody or antibody fragment.

24. (canceled)

25. A pharmaceutical composition comprising the antibody or antibody fragment of claim 1.

26. A nucleic acid encoding the antibody or antibody fragment of claim 1.

27. A method of preparing an antibody or antibody fragment of claim 1, the method comprising expressing a nucleic acid encoding the antibody or antibody fragment in a cell.

28. A cell comprising the nucleic acid of claim 26.

29. A polypeptide comprising
(a) SEQ ID NO: 192 or SEQ ID NO: 193;
(b) a fragment of five or more contiguous amino acids of SEQ ID NO: 192 or SEQ ID NO: 193; or
(c) a combination thereof;
wherein the polypeptide has fewer than 50 total amino acids and the polypeptide inhibits binding of an antibody or antibody fragment of claim 1 to C3d.

30. A nucleic acid encoding the polypeptide of claim 29.

31. A method of preparing an anti-C3d antibody comprising immunizing an animal with a polypeptide of claim 29, or screening for an antibody that specifically binds to a polypeptide of claim 29.

32-36. (canceled)
